(19) United States
(12) Patent Application Publication
(10) Pub. No.: US 2026/0097178 A1
(43) Pub. Date: Apr. 9, 2026

(54) AIRWAY MANAGEMENT SYSTEM

(71) Applicant: Ming YANG, Tianjin (CN)

(72) Inventor: Ming YANG, Tianjin (CN)

(21) Appl. No.: 19/185,225

(22) Filed: Apr. 21, 2025

Related U.S. Application Data
(63) Continuation of application No. PCT/CN2023/122380, filed on Sep. 28, 2023.

(30) Foreign Application Priority Data
Nov. 15, 2022 (CN) 202211428717.7

(51) Int. Cl. A61M 16/06; A61M 16/01; A61M 16/04; A61M 16/08; A61M 39/00; A61M 39/10

(52) U.S. Cl. CPC A61M 16/06; A61M 16/01; A61M 16/042; A61M 16/0875; A61M 16/0891; A61M 39/10; A61M 2039/0009; A61M 2039/1027; A61M 2202/0007; A61M 2202/0241; A61M 2210/065; A61M 2210/1032

(57) ABSTRACT

An airway management system, comprising: a breathing circuit connector component, an inner tube component, and a connecting tube, further comprising: a laryngeal mask component and a telescopic tube component; or, an endotracheal tube component, the breathing circuit connector component comprises a breathing circuit connector; a plurality of cavities is provided in the laryngeal mask airway tube of the laryngeal mask component; a plurality of cavities is provided in a endotracheal tube of the endotracheal tube component; and one end of the breathing circuit connector is connected to a laryngeal mask connector of the laryngeal mask component, or an endotracheal tube connector of the endotracheal tube component, or the laryngeal mask connector and an inner tube connector of the inner tube component, or the endotracheal tube connector and the inner tube connector, or a telescopic tube machine end connector of the telescopic tube component and the inner tube connector.

### TECHNICAL FIELD

This invention relates to the field of clinical medical device technology, specifically relating to an airway management system.

### BACKGROUND

An airway management system will be typically used on patients during the administration of general anesthesia or during treatment in an intensive care unit (ICU). The airway management system typically comprises an anaesthetic mask, a laryngeal mask, an endotracheal tube, a connecting tube, a telescopic tube, a heat and moisture exchanger (HME), a breathing circuit and the like. The airway management system can achieve functions such as general anesthesia, respiratory support, detection of inhaled or exhaled gas composition, delivery of oxygen and anesthetic gases, and discharge of respiratory waste gases. The respiratory patterns for the patients include: both spontaneous respiration and apparatus-controlled ventilation.

Due to the influence of own constitutions of the patients, drugs used and underlying diseases, there are significant differences in tidal volume, respiratory rate, and minute ventilation of patients who retain spontaneous respiration. Smaller tidal volume, respiratory rate, minute ventilation, and airway pressure should be selected as possible for patients with apparatus-controlled ventilation on the premise of satisfying gas exchange. Every time the patient inhales, he/she will inhale part of the exhaled gas. Moreover, the same patient often experiences the two respiratory patterns. An existing airway management system has a common space for the inhaled gas and the exhaled gas, which cannot meet various different respiratory patterns of the patients. When the tidal volume of the spontaneous respiration of the patient is high, the airway resistance will increase. When the tidal volume of the spontaneous respiration of the patient is small or protective apparatus-controlled ventilation is administrated, ineffective ventilation will increase, and repeated inhalation is more frequent, which influences gas exchange. A patient with severe pneumonia requires invasive ventilation due to fact that an increase in anatomic dead space results in a decrease in blood oxygen content and an increase in blood carbon dioxide content, that is, the patient is connected to a ventilator via the breathing circuit after tracheal intubation. There are several reasons for the increase in anatomic dead space of the patient: 1. inflammatory exudates fill alveoli; 2. the blood flow ratio for supine position ventilation is imbalanced; and 3. apparatus ventilation causes the increase in anatomic dead space. An existing solution is prone position ventilation for the patients, and the prone position ventilation can reduce the anatomic dead space of the patient, and corrects the imbalance of the blood flow ratio of ventilation. Prone position ventilation treatment is not commonly used in clinical practice due to the following drawbacks: 1. medical and nursing resources are overstretched; 2. the risk of airway obstruction is increased; and 3. the risk of pressure sores and head and face injuries in patients are increased.

Due to the limitation of the internal diameter of a ventilation tube, only an endotracheal tube with an internal diameter of less than 7.0 mm can be inserted into a conventional intubating laryngeal mask. An endotracheal tube with an average internal diameter of 7.5 mm should be selected for female patients in China, and an endotracheal tube with an average internal diameter of 8.5 mm should be selected for male patients in China. If the endotracheal tube with the internal diameter of less than 7.0 mm is selected, there may be the following hazards in clinical practice: 1. the artificial airway resistance is increased, which increases respiratory and circulatory system complications; 2. The cuff pressure of the endotracheal tube is increased, which causes injure to the mucosa of a tracheal lining. 3. Operations by a ventilation cavity of the endotracheal tube will be limited, comprising insertion of a bronchial occluder, examination and treatment under a fiberoptic bronchoscope, etc.

The tail end of a conventional gas-sampling line is provided in an apparatus dead space of an artificial airway, and its position is not fixed. Since a sampling site is far away from alveoli and interfered by the exhaled exhaust gas of the patient, a numerical value obtained by sampling analysis is delayed and inaccurate.

A conventional inner tube has a smooth outer wall and is cylindrical, and is prone to adherence after being inserted into the laryngeal mask and a lumen of the endotracheal tube, thereby causing obstruction between inner and outer tubes.

A conventional connecting tube is of an equal diameter structure and cannot be connected to a patient end of a breathing circuit connector in this invention.

A conventional breathing circuit connector is L-shaped, Y-shaped or used in combination, and cannot be applied in this invention.

### SUMMARY

Patients typically use an airway management system during general anesthesia implementation or during treatment in the Intensive Care Unit (ICU). The airway management system typically comprises an anaesthetic mask, a laryngeal mask, an endotracheal tube, a connecting tube, a telescopic tube, a heat and moisture exchanger (HME), a breathing circuit and the like. The airway management system can achieve functions such as general anesthesia, respiratory support, detection of inhaled or exhaled gas components, delivery of oxygen and anesthetic gases, and discharge of respiratory waste gases. The respiratory patterns for the patients include two types: spontaneous respiration and apparatus-controlled ventilation. A patient end of a gas sampling cavity of the laryngeal mask component or the endotracheal tube component and the gas sampling cavity are located on the ventral side of the patient, and the patient end of the gas sampling cavity is close to a patient end of a tube, which makes sampling analysis more sensitive and accurate. A patient end of a throat aspiration cavity of the laryngeal mask component or the endotracheal tube component and the throat aspiration cavity are located on the dorsal side of the patient; the patient end of the throat aspiration cavity is close to the patient end of the catheter; and the throat aspiration cavity can aspirate secretions near the glottis of the patient and or may serve as a standby gas sampling cavity. The gas sampling cavity and the throat aspiration cavity are fixedly mated, and are symmetrically provided, which guarantees the continuity and reliability of gas sampling analysis. The laryngeal mask airway connector can be connected to a patient end of a telescopic tube component; when the telescopic tube component is fully extended to its completely open state, the inner tube component can be inserted via the laryngeal mask component and the telescopic tube component; a patient end of the inner tube component is located on the glottis, and in a mask body of the laryngeal mask component, and is aligned with a patient end of a laryngeal mask airway tube, which can eliminate apparatus dead space, avoid that a patient inhales his/her own exhaled exhaust gas, and can improve the sensitivity and accuracy of results of gas sampling analysis in the airway of the patient. As the telescopic tube component gradually contracts, the patient end of the inner tube component moves towards the lungs of the patient, passes through the glottis, and enters the trachea; and when the telescopic tube component contracts until it is in its original state, the patient end of the inner tube component reaches the trachea, thereby eliminating all the apparatus dead space and some of anatomic dead space. The inner tube connector and the telescopic tube machine end connector are jointly connected to the patient end of the breathing circuit connector, and the breathing circuit connector is connected to an anesthesia machine or a ventilator through a breathing circuit.

The purpose of the first aspect of this invention is to provide an airway management system to solve the technical problem of difficulty in eliminating apparatus dead space in existing system.

The airway management system provided in the first aspect of this invention comprising a laryngeal mask component and an inner tube component, the inner tube component comprises an inner tube body, and a third ventilation cavity is arranged inside the inner tube body; where the laryngeal mask component comprises a mask body, a laryngeal mask airway tube connected to one end of the mask body, a first ventilation cavity is arranged inside the laryngeal mask airway tube, and the first ventilation cavity is configured for inserting the inner tube component, after inserting the inner tube component, between the first ventilation cavity and the third ventilation cavity, one forms an inhalation cavity and another forms an exhalation cavity;

The patient end of the inner tube component is configured to be positioned within the mask body, and aligned with the patient end of the laryngeal mask airway tube; The patient end of the inner tube component can also be configured to enter the trachea of the patient through the glottis of the patient.

The beneficial effects of the airway management system provided in this invention are:

By inserting the inner tube component into the laryngeal mask component, the inner tube connector and the laryngeal mask airway connector jointly connect to the patient end of the breathing circuit connector, where the breathing circuit connector is connected to an anesthesia machine or a ventilator through a breathing circuit, which can eliminate apparatus dead space and avoid that the patient inhales his/her own exhaled exhaust gas.

The apparatus dead space and some of the anatomic dead space are eliminated, which has clinical significance for patients who require low tidal volume and low-driving pressure ventilation. The patients who require low tidal volume and low-driving pressure ventilation comprise but are not limited to: 1 patients undergoing pediatric surgery; 2 patients undergoing laparoscopic surgery that require carbon dioxide filling; 3 patients undergoing thoracic surgery; 4 patients suffering from pulmonary diseases and undergoing general anesthesia; 5 patients undergoing massive transfusion and infusion surgery; 6 patients undergoing prolonged surgery; 7 patients with chronic obstructive pulmonary disease (COPD) or acute respiratory distress syndrome (ARDS); and 8 patients with severe pneumonia who need invasive ventilation treatment.

For ARDS patients, this application can achieve a good therapeutic effect in the supine position, and will achieve a better therapeutic effect if combined with the prone position ventilation treatment.

Different specifications of laryngeal mask components or endotracheal tube components of this application can be used in combination with different specifications of inner tube components to meet the needs of patients with different glottic areas and different ventilator parameter settings.

In one embodiment, between the ventilation cavity formed after the first ventilation cavity inserted into the inner tube component and the third ventilation cavity, one forms an inhalation cavity and another forms an exhalation cavity; or

between the part of the first ventilation cavity inserted into the inner tube component and the third ventilation cavity, one forms an inhalation cavity and another forms an exhalation cavity.

In one embodiment, the laryngeal mask component further comprises a laryngeal mask airway connector, where the other end of the laryngeal mask airway tube is connected to the laryngeal mask airway connector; The inner tube component also comprises an inner tube connector connected to the inner tube body; The airway management system also comprises a breathing circuit connector component; the breathing circuit connector component comprises a breathing circuit connector, a breathing circuit connector top end cover, and a first Luer taper; one end of the breathing circuit connector is connected to the laryngeal mask airway connector, or connected to the laryngeal mask airway connector and the inner tube connector, and the other end of the breathing circuit connector is configured to connect to an anesthesia machine or a ventilator through a breathing circuit;

The breathing circuit connector comprises a patient end of the breathing circuit connector and a machine end of the breathing circuit connector; the patient end of the breathing circuit connector comprises a patient end inner connector and a patient end outer connector, wherein the patient end outer connector is configured to connect to the laryngeal mask airway connector, the patient end inner connector is configured to connect to the inner tube connector.

The laryngeal mask airway connector is connected to the patient end of the breathing circuit connector, and at this time, the breathing circuit connector top end cover is placed on the machine end first inner connector of the breathing circuit connector; By inserting a machine end second inner connector into the inner tube body, the inner tube connector of the inner tube component and the machine end outer connector of the breathing circuit connector can be connected to the patient end of the breathing circuit respectively, where the machine end of the breathing circuit is connected to an anesthesia machine or a ventilator; as the inner tube body is gradually inserted, the apparatus dead space in the airway management system gradually decrease; when the patient end of the inner tube component is located in the mask body of the laryngeal mask component, and is aligned with the patient end of the laryngeal mask airway tube, the apparatus dead space can be eliminated; at this time, the patient end of the inner tube component passes through the glottis of the patient and enters the trachea of the patient, thereby eliminating all the apparatus dead space and some of the anatomic dead space.

The laryngeal mask airway tube and the laryngeal mask airway connector have internal diameters larger than that of the existing product, which can reduce the airway resistance, implant a diagnosis and treatment device with a larger external diameter and insert an endotracheal tube with a larger external diameter via the laryngeal mask airway tube and the laryngeal mask airway connector. A sampling site of the laryngeal mask component is close to the patient end of the catheter, which can make the sampling site close to the alveoli to the greatest extent, such that the sampling analysis is more sensitive and accurate, and a sampling analysis numerical value and an arterial blood gas analysis numerical value are more approximate and have good correlation.

In one embodiment, the airway management system further comprises a telescopic tube component, the telescopic tube component comprises a telescopic tube patient end connector, a telescopic tube machine end connector, and a telescopic tube body, one end of the telescopic tube body is connected to the telescopic tube patient end connector, the other end of the telescopic tube body is connected to the telescopic tube machine end connector; the telescopic tube patient end connector is configured to connect the laryngeal mask airway connector; the inner tube component is configured to be inserted via the laryngeal mask airway tube, the laryngeal mask airway connector and the telescopic tube component; the telescopic tube machine end connector and the inner tube connector are configured to connect to the patient end of the breathing circuit connector;

When the telescopic tube component is configured to fully extend to its completely open state, the patient end of the inner tube component reaches the trachea of the patient; when the telescopic tube component gradually contracts, the patient end of the inner tube component moves towards the lungs of the patient, passes through the glottis of the patient, and enters the trachea of the patient.

The laryngeal mask airway connector can be connected to the patient end of the telescopic tube component; when the telescopic tube component is fully extended to its completely open state; the inner tube component can be inserted via the laryngeal mask component and the telescopic tube component; the patient end of the inner tube component is located on the glottis of the patient, and in the mask body of the laryngeal mask component, and is aligned with the patient end of the laryngeal mask airway tube, the inner tube connector and the telescopic tube machine end connector are jointly connected to the patient end of the breathing circuit connector, and the breathing circuit connector is connected to the anesthesia machine or the ventilator through the breathing circuit, which can eliminate apparatus dead space, avoid that a patient inhales his/her own exhaled exhaust gas from the laryngeal mask component, and can improve the sensitivity and accuracy of gas sampling analysis in the airway of the patient. As the telescopic tube component gradually contracts, the patient end of the inner tube component moves towards the lungs of the patient, passes through the glottis of the patient, and enters the trachea; when the telescopic tube component contracts until it is in its original state, the patient end of the inner tube component can be made reach the trachea of the patient, thereby eliminating all the apparatus dead space and some of anatomic dead space; and the inner tube connector and the telescopic tube machine end connector are jointly connected to the patient end of the breathing circuit connector, and the breathing circuit connector is connected to the anesthesia machine or the ventilator through the breathing circuit.

After the inner tube component is inserted via the laryngeal mask component and telescopic tube component, the patient end of the inner tube component can reach the trachea of the patient. Compared to an existing laryngeal mask product, a third ventilation cavity of the inner tube component can be selected as an inhalation channel, and a gap between the inner tube body and the trachea, the glottis, and the laryngeal mask airway tube may serve as an exhalation channel, which guarantees smooth respiration, reduces airway pressure, and lowers the risk of accidental aspiration. Due to the ventilation and positioning function of the inner tube component, the risk of airway obstruction is eliminated, and the stability of an artificial airway is increased. Compared to an existing endotracheal tube product, the risk of the mucosa of a tracheal lining is compressed after the cuff is inflated to cause mucosal ischemic necrosis is eliminated.

In an embodiment, the telescopic tube component further comprises: annular protrusions and a stopper; the annular protrusions are provided on outer side walls of the telescopic tube patient end connector and the telescopic tube machine end connector, respectively; the stopper is configured to limit the telescopic tube component; and when the telescopic tube component is fully extended to its completely open state, the stopper is located between the annular protrusions.

The laryngeal mask airway connector can be connected to the patient end of the telescopic tube component; when the telescopic tube component is fully extended to its completely open state, an optional way is to place the stopper between the annular protrusions; the inner tube component can be inserted via the laryngeal mask component and the telescopic tube component; the patient end of the inner tube component is located on the glottis of the patient, and in the mask body of the laryngeal mask component, and is aligned with the patient end of the laryngeal mask airway tube, the inner tube connector and the telescopic tube machine end connector are jointly connected to the patient end of the breathing circuit connector, and the breathing circuit connector is connected to the anesthesia machine or the ventilator through the breathing circuit, which can eliminate apparatus dead space, avoid that a patient inhales his/her own exhaled exhaust gas, and can improve the sensitivity and accuracy of gas sampling analysis in the airway of the patient. The annular protrusions of the telescopic tube component of this invention are provided on the outer side walls of the telescopic tube patient end connector and the telescopic tube machine end connector, respectively; the stopper is configured to limit the telescopic tube component; and when the telescopic tube component is fully extended to its completely open state, the stopper is located between the annular protrusions, so that the telescopic tube component cannot contract. This avoids that the patient end of the inner tube component injures the glottis and trachea of the patient due to the fact that the telescopic tube component contracts. After the stopper is removed, as the telescopic tube component gradually contracts, the patient end of the inner tube component moves towards the lungs of the patient, passes through the glottis of the patient, and enters the trachea; when the telescopic tube component contracts until it is in its original state, the patient end of the inner tube component can be made reach the trachea of the patient, thereby eliminating all the apparatus dead space and some of anatomic dead space; and the inner tube connector and the telescopic tube machine end connector are jointly connected to the patient end of the breathing circuit connector, and the breathing circuit connector is connected to the anesthesia machine or the ventilator through the breathing circuit.

The laryngeal mask component is connected to the patient end of the breathing circuit connector, and at this time, the breathing circuit connector top end cover is placed on the machine end first inner connector of the breathing circuit connector; the inner tube body of the inner tube component is inserted through the machine end second inner connector of the breathing circuit connector, so that the inner tube connector and the machine end outer connector are each connected to the anesthesia machine or the ventilator through the breathing circuit; the laryngeal mask airway connector of the laryngeal mask component is connected to the patient end of the breathing circuit connector, and the inner tube body is inserted through the machine end second inner connector; as the inner tube body is gradually inserted, the apparatus dead space in the airway management system gradually decrease; when the patient end of the inner tube component is located in the mask body of the laryngeal mask component, and is aligned with the patient end of the laryngeal mask airway tube, the apparatus dead space can be eliminated; at this time, the patient end of the inner tube component passes through the glottis of the patient and enters the trachea of the patient, thereby eliminating all the apparatus dead space and some of the anatomic dead space. This method allows for the dead space volume of the airway management system to be continuously adjustable from being greater than that of the existing product to eliminating all the apparatus dead space plus some of the anatomic dead space, and is suitable for treating patients with hyperventilation.

In one embodiment, a first gas sampling cavity, and a first throat aspiration cavity are further provided in the laryngeal mask airway tube.

In one embodiment, a drainage cavity is further provided in the laryngeal mask airway tube.

In one embodiment, a video component cavity is further provided in the laryngeal mask airway tube.

In one embodiment, a standby reserved cavity is further provided in the laryngeal mask airway tube.

In one embodiment, the patient end of the first gas sampling cavity and the patient end of the first throat aspiration cavity are close to the patient end of the laryngeal mask airway tube, the first gas sampling cavity inside the laryngeal mask airway tube and the first throat aspiration cavity inside the laryngeal mask airway tube are fixedly mated.

The patient end of the first gas sampling cavity of the laryngeal mask component and the first gas sampling cavity are located on the ventral side of the patient. The patient end of the first gas sampling cavity is close to the patient end of the catheter, which can improve the sensitivity and accuracy of gas sampling analysis in the airway of the patient. The patient end of the first throat aspiration cavity of the laryngeal mask component and the throat aspiration cavity are located on the dorsal side of the patient; the patient end of the first throat aspiration cavity is close to the patient end of the laryngeal mask airway tube; and the first throat aspiration cavity can aspirate secretions near the glottis of the patient and or may serve as a standby gas sampling cavity. The patient end of the first gas sampling cavity, the first gas sampling cavity and the first throat aspiration cavity are fixedly mated, and are symmetrically provided, which guarantees the continuity and reliability of gas sampling analysis.

The first gas sampling cavity of the laryngeal mask component can be provided in the tube of the laryngeal mask airway tube, or may be independent outside the laryngeal mask airway tube. The first throat aspiration cavity and the first gas sampling cavity are symmetrically positioned and have identical structures. The patient end of the first throat aspiration cavity and the patient end of the first gas sampling chamber are close to the patient end of the catheter, symmetrically positioned, and have identical structures. The first throat aspiration cavity can be used to suction pharyngeal secretions, and can also serve as a standby sampling cavity when the gas sampling cavity is blocked, ensuring the continuity and reliability of gas sampling analysis.

The purpose of the second aspect of this invention is to provide an airway management system to solve the technical problem of difficult elimination of apparatus dead space.

The airway management system provided in the second aspect of this invention comprises an endotracheal tube component and an inner tube component, wherein the inner tube component comprises an inner tube body, a third ventilation cavity is arranged inside the inner tube body. The endotracheal tube component comprises an endotracheal tube, a second ventilation cavity is arranged in the endotracheal tube. The second ventilation cavity is configured for inserting the inner tube component; after inserting the inner tube component, between the second ventilation cavity and the third ventilation cavity, one forms an inhalation cavity and another forms an exhalation cavity. The endotracheal tube has the same internal diameter. The patient end of the inner tube component is configured to be align with the patient end of the endotracheal tube. The patient end of the inner tube component is also configured to enter the trachea of the patient through the patient end of the endotracheal tube.

The beneficial effects brought by the airway management system of this invention are:

By inserting the endotracheal tube component into the inner tube component, the inner tube connector and the endotracheal tube connector jointly connect to the patient end of the breathing circuit connector, where the breathing circuit connector is connected to an anesthesia machine or a ventilator through a breathing circuit, which can eliminate apparatus dead space and avoid that the patient inhales his/her own exhaled exhaust gas.

Endotracheal tube components of different specifications can be used in combination with inner tube components of different specifications to meet the needs of patients with different glottal areas and different ventilator parameter settings.

The purpose of the third aspect of this invention is to provide an airway management system to solve the technical problem of difficult elimination of apparatus dead space.

The airway management system provided in the third aspect of this invention comprises an endotracheal tube component and an inner tube component, wherein the inner tube component comprises an inner tube body, a third ventilation cavity is arranged inside the inner tube body. The second ventilation cavity is configured for inserting the inner tube component; after inserting the inner tube component, between the second ventilation cavity and the third ventilation cavity, one forms an inhalation cavity and another forms an exhalation cavity. The internal diameter of the endotracheal tube is configured such that the internal diameter of the segment located below the glottis of a patient when in use is smaller than that of the segment located above the glottis of the patient when in use. The patient end of the inner tube component is configured to be located at the patient end of the large diameter portion of the endotracheal tube of the unequal diameter structure.

The beneficial effects brought by the airway management system of this invention are:

The internal diameter of the endotracheal tube in this invention could be configured such that the internal diameter of the segment located below the glottis of a patient when in use is smaller than that of the segment located above the glottis of the patient when in use. This can avoid injure to the patient's glottis and trachea, improve patient comfort and reduce complications.

In one embodiment, the ventilation cavity formed after the second ventilation cavity is inserted into the inner tube component and the third ventilation cavity, one forms an inhalation cavity and another forms an exhalation cavity; or

the portion of the second ventilation cavity inserted into the inner tube component and the third ventilation cavity, one forms an inhalation cavity, and another forms an exhalation cavity.

In one embodiment, the endotracheal tube component comprises an endotracheal tube connector. The inner tube component comprises an inner tube connector connected to the inner tube body. The airway management system further comprises a breathing circuit connector component, the breathing circuit connector component comprises a breathing circuit connector, one end of the breathing circuit connector is connected to the endotracheal tube connector or to the endotracheal tube connector and the inner tube connector, the other end of the breathing circuit connector is configured to be connected to the anesthesia machine or ventilator via breathing circuit.

The breathing circuit connector comprises a patient end and a machine end of the breathing circuit connector. The patient end of the breathing circuit connector comprises a patient end inner connector and a patient end outer connector. The patient end outer connector is configured to be connected to the endotracheal tube connector, the patient end inner connector is configured to be connected to the inner tube connector.

The endotracheal tube, the endotracheal tube connector of this invention have internal diameters larger than that of the existing product, a diagnosis and treatment device with a larger external diameter can be implanted. A sampling site of the endotracheal tube component is close to the patient end of the catheter, which can make the sampling site close to the alveoli to the greatest extent, such that the sampling analysis is more sensitive and accurate, and a sampling analysis numerical value and an arterial blood gas analysis numerical value are more approximate and have good correlation.

In one embodiment, the machine end of the breathing circuit connector comprises a machine end first inner connector, a machine end second inner connector, and a machine end outer connector. The machine end of the breathing circuit connector is provided with three channels, the three channels being a machine end first inner channel, a machine end second inner channel, and a machine end outer channel. The patient end of the breathing circuit connector comprises a patient end inner channel, and a patient end outer channel. The machine end first inner channel and the machine end second inner channel is connected to the patient end inner channel, the machine end outer channel is connected to the patient end outer channel. The patient end inner channel and the patient end outer channel are of a coaxial inner and outer dual-channel structure. The machine end first inner connector and the machine end outer connector or the machine end second inner connector and the machine end outer connector are connected to the anesthesia machine or the ventilator through the breathing circuit, respectively.

After the laryngeal mask airway connector or the endotracheal tube connector is connected to the patient end of the breathing circuit connector component, the breathing circuit connector component can rotated in any direction within a circular range, facilitating the insertion and removal of the laryngeal mask airway connector of the laryngeal mask component or the endotracheal tube connector of the endotracheal tube component with the patient end of the breathing circuit connector component, and facilitating the placement of the breathing circuit.

The breathing circuit connector component employs a dual-channel design at the patient end and a three-channel design at the machine end, which can guarantee that the laryngeal mask component or the endotracheal tube component is implanted into the patient. After the laryngeal mask airway connector of the laryngeal mask component or the endotracheal tube connector of the laryngeal mask component is connected to the patient end of the breathing circuit connector component, the inner tube component is implanted under the guidance of a fiberoptic bronchoscope via the machine end second inner connector of the breathing circuit connector without interrupting low dead space volume ventilation. The patient end of the first Luer taper of the breathing circuit connector component of this invention can be connected to the gas-sampling line, and the gas-sampling line can collect gas inside the laryngeal mask airway tube, the endotracheal tube, on the glottis or inside the trachea for detection.

In one embodiment, the breathing circuit connector component further comprises a breathing circuit connector top end cover. The openable breathing circuit connector top end cover is provided at the top end of the machine end second inner connector. The breathing circuit connector top end cover is configured to be connected to the machine end first inner connector.

The laryngeal mask airway connector is connected to the patient end of the breathing circuit connector, or the endotracheal tube connector of the endotracheal tube component in which internal diameters are equal is connected to the patient end of the breathing circuit connector, and at this time, the breathing circuit connector top end cover is placed on the machine end first inner connector.

The endotracheal tube component can be connected to the patient end of the breathing circuit connector, and at this time, the breathing circuit connector top end cover is placed on the machine end first inner connector. By inserting the inner tube body into the machine end second inner connector, so that the inner tube connector and the machine end outer connector are each connected to the patient end of the breathing circuit, and the machine end of the breathing circuit is connected to the anesthesia machine or the ventilator. The endotracheal tube connector of the endotracheal tube component in which internal diameters are equal is connected to the patient end of the breathing circuit connector and after the machine end second inner connector of the breathing circuit connector is inserted into the inner tube body, as the inner tube body is gradually inserted, the apparatus dead space in the airway management system gradually decrease, when the patient end of the inner tube component and the patient end of the endotracheal tube are aligned, apparatus dead space is eliminated; the patient end of the inner tube component is further inserted and enters the trachea of the patient, thereby eliminating all the apparatus dead space and some of the anatomic dead space.

The endotracheal tube connector of the endotracheal tube component of a structure in which internal diameters are unequal is connected to the patient end of the breathing circuit connector, and the machine end second inner connector is inserted into the inner tube body; as the inner tube body is gradually inserted, the apparatus dead space in the airway management system gradually decrease; and when the patient end of the inner tube component is located at the patient end of a large internal diameter portion of the endotracheal tube of the unequal diameter structure, some of the apparatus dead space can be eliminated.

In one embodiment, the breathing circuit connector component further comprises a first Luer taper; and the middle of the breathing circuit connector top end cover is connected to the first Luer taper.

In one embodiment, the airway management system further comprises a connecting tube, and the connecting tube is of an unequal diameter structure. The connecting tube comprises a connecting tube body. When clinical anesthesia induction is performed, one end of the connecting tube body is configured to be connected to the patient end of the breathing circuit connector, and the other end of the connecting tube body is configured to be connected to the anesthetic mask.

In one embodiment, the connecting tube further comprises a second Luer taper. The second Luer taper is configured to be connected to the gas-sampling line of the monitor.

The connecting tube of this application is designed as the unequal diameter structure, and the second Luer taper of the connecting tube can serve as a gas sampling site during anesthesia induction and anesthesia maintenance. The patient end of the connecting tube can be connected to an existing mask, an existing laryngeal mask, the existing endotracheal tube, the laryngeal mask component of this application, the endotracheal tube component of this application, the laryngeal mask component and the inner tube component of this application, and the endotracheal tube component and the inner tube component of this application, and the machine end of the connecting tube is connected to the patient end of the breathing circuit connector component. After connection, the apparatus dead space can be enlarged, which is suitable for the induction stage of general anesthesia and the treatment of patients with hyperventilation.

In one embodiment, an outer wall of the inner tube body is provided with a plurality of protrusions; the protrusion is configured for positioning between the inner tube body and the first ventilation cavity or the second ventilation cavity.

There are several protrusions symmetrically designed in the axial direction on the outer wall of the inner tube body of the inner tube component in this invention, and after inserting into the ventilation cavity of the laryngeal mask airway tube or endotracheal tube, the inner tube body can be kept at the axis center position of the ventilation cavity into which the inner tube body is inserted under the support of the protrusions, which guarantees that the lumen of the ventilation cavity between the inner tube body and the laryngeal mask airway tube or the endotracheal tube is unimpeded. The inner tube component is of a detachable structure, and inner tube components of different internal diameters, external diameters, and lengths can be selected according to the anatomical characteristics of the throat and trachea of the patient and clinical use needs.

In one embodiment, a second throat aspiration cavity, a second gas sampling cavity, and a cuff inflation cavity are provided in the endotracheal tube.

In one embodiment, the patient end of the second gas sampling cavity and the patient end of the second throat aspiration cavity are close to the patient end of the endotracheal tube. The second gas sampling cavity and the second throat aspiration cavity are fixedly mated.

The patient end of the second gas sampling cavity of the endotracheal tube component and the gas sampling cavity are located on the ventral side of the patient. The patient end of the second gas sampling cavity is close to the patient end of the catheter, which can improve the sensitivity and accuracy of gas sampling analysis in the airway of the patient. The patient end of the second throat aspiration cavity of the endotracheal tube component and the throat aspiration cavity are located on the dorsal side of the patient; the patient end of the second throat aspiration cavity is close to the patient end of the catheter; and the second throat aspiration cavity can aspirate secretions near the glottis of the patient and or may serve as a standby gas sampling cavity. The patient end of the second gas sampling cavity, the patient end of the second gas sampling cavity and of the second throat aspiration cavity, the second throat aspiration cavity are fixedly mated, and are symmetrically provided, which guarantees the continuity and reliability of gas sampling analysis.

The second gas sampling cavity of the endotracheal tube component of this invention can be provided in the tube of the endotracheal tube, or may be independent outside the endotracheal tube, thus facilitating the design, research and development, and production of products.

### BRIEF DESCRIPTION OF THE DRAWINGS

The above and other objects, features and advantages of the exemplary embodiments of this invention will become readily understood by reading the following detailed description with reference to the accompanying drawings. In the accompanying drawings, several embodiments of this invention are illustrated in an exemplary rather than restrictive manner.

In the accompanying drawings, the same or corresponding reference numerals represent the same or corresponding parts.

FIG. 1 is an assembly schematic diagram of the laryngeal mask component and the inner tube component in this invention;

FIG. 2 is a structural schematic diagram of the laryngeal mask component in embodiment 1 in this invention;

FIG. 3 is a top view of FIG;

FIG. 3a is a cross-sectional schematic diagram along line A-A in FIG. 3;

FIG. 4 is a structural schematic diagram of an inner tube component in this invention;

FIG. 4a is a cross-sectional schematic diagram along line E-E;

FIG. 5 is an assembly schematic diagram of the laryngeal mask component, the inner tube component and the breathing circuit connector component in this invention;

FIG. 6 is an assembly-connection schematic diagram of the laryngeal mask component and the breathing circuit connector component in this invention;

FIG. 7 is a structural schematic diagram of the breathing circuit connector component in this invention;

FIG. 8 is a top view of FIG. 7;

FIG. 9 is a cross-sectional schematic diagram along line B-B in FIG. 7;

FIG. 10 is a cross-sectional schematic diagram along line C-C in FIG. 8;

FIG. 11 is a cross-sectional schematic diagram along line D-D in FIG. 10;

FIG. 12 is an assembly schematic diagram of a laryngeal mask component, an inner tube component and a telescopic tube component in an original state in this application;

FIG. 13 is an assembly schematic diagram of the laryngeal mask component, the inner tube component and the telescopic tube component in a fully extended state in this application;

FIG. 14 is a schematic diagram of the telescopic tube component in an original state in this application;

FIG. 15 is a schematic diagram of the telescopic tube component in a fully extended state in this application;

FIG. 15a is a structural schematic diagram of the telescopic tube component with a stopper;

FIG. 16 is an assembly schematic diagram of the laryngeal mask component, and the inner tube component, the breathing circuit connector component, and the telescopic tube component in this invention;

FIG. 17 is a structural schematic diagram of Embodiment 2 of this application;

FIG. 17a is a cross-sectional schematic diagram along line F-F in FIG. 17;

FIG. 18 is a structural schematic diagram of Embodiment 3 of this application;

FIG. 18a is a cross-sectional schematic diagram along line G-G in FIG. 18;

FIG. 19 is an assembly schematic diagram of an endotracheal tube component and an inner tube component of Embodiment 4 of this application;

FIG. 19a is a cross-sectional schematic diagram along line H-H in FIG. 19;

FIG. 19b is a cross-sectional schematic diagram along line I-I in FIG. 19;

FIG. 19c is a view in the direction of m in FIG. 19;

FIG. 20 is a top view of FIG. 19;

FIG. 21 is an assembly schematic diagram of the endotracheal tube component, the inner tube component and the breathing circuit connector component of embodiment 4 of this invention;

FIG. 22 is an assembly schematic diagram of the endotracheal tube component and the inner tube component of Embodiment 5 of this application;

FIG. 22a is a cross-sectional schematic diagram along line J-J in FIG. 22;

FIG. 22b is a cross-sectional schematic diagram along line K-K in FIG. 22;

FIG. 22c is a cross-sectional schematic diagram along line L-L in FIG. 22;

FIG. 23 is a top view of FIG. 22;

FIG. 24 is an assembly schematic diagram of the endotracheal tube component, the inner tube component and the breathing circuit connector component of embodiment 5 of this application;

FIG. 25 is a structural schematic diagram of the connecting tube in this application;

FIG. 25a is a top view of FIG. 25.

### REFERENCE NUMERALS

100. Laryngeal mask component; 101. Mask body; 102. laryngeal mask airway tube; 103. Laryngeal mask airway connector; 104. Laryngeal mask inflation tube; 105. Laryngeal mask inflation valve; 106. First ventilation cavity; 107. First throat aspiration cavity; 108. First gas sampling cavity; 109. Video component cavity; 110. Drainage cavity; 111. Standby reserved cavity; 112. First gas-sampling line; 113. First throat aspiration tube; and 114. Third Luer taper;
200. Endotracheal tube component; 201. Cuff; 202. Endotracheal tube; 203. Endotracheal tube connector; 204. Cuff inflation tube; 205. Cuff inflation valve; 206. Second ventilation cavity; 207. Second throat aspiration cavity; 208. Second gas sampling cavity; 209. Second gas-sampling line; 210. Second throat aspiration tube; 211. Fourth Luer taper; and 212. Cuff inflation cavity;
300. Breathing circuit connector component; 310. Breathing circuit connector; 311. Patient end inner channel; 312. Patient end outer channel; 313. Machine end first inner channel; 314. Machine end second inner channel; 315. Machine end outer channel; 316. Patient end inner connector; 317. Patient end outer connector; 318. Machine end first inner connector; 319. Machine end second inner connector; 3110. Machine end outer connector; 320. Breathing circuit connector top end cover; and 330. First Luer taper;
400. Inner tube component; 401. Inner tube body; 402. Third ventilation cavity; 403. Inner tube connector; and 404. protrusion;
500. Connecting tube; 501. Connecting tube body; 502. Second Luer taper;
600. Telescopic tube component; 601. Telescopic tube body; 602. Telescopic tube patient end connector; 603. Telescopic tube machine end connector; 604. Stopper; and 605. Annular protrusion.

### DETAILED DESCRIPTION

In order to make the objects, technical solutions and advantages of this application more clearly understood, this application is further described in detail below in conjunction with the embodiments and the accompanying drawings. The described embodiments are merely some rather than all of the embodiments of this application. All other embodiments obtained by those skilled in the art based on the embodiments in this application without making creative efforts fall within the scope of protection of this application. It should be understood that the specific embodiments described here are only intended to explain this application and are not intended to limit this application.

In the descriptions of this specification, descriptions of reference terms such as “one embodiment”, “some embodiments”, “an embodiment”, “a specific embodiment” or “some embodiments” means that specific features, structures, materials, or characteristics described with reference to the embodiment or the embodiment are comprised in at least one embodiment or embodiment of this application. Moreover, the described specific features, structures, materials, or characteristics can be combined in a proper manner in any one or more embodiments or embodiments. Furthermore, without contradictory, those skilled in the art can integrate and combine different embodiments or embodiments described in this specification and the features of different embodiments or embodiments.

Furthermore, the terms “first”, “second”, “third”, and “fourth” are only used for descriptive purposes and cannot be understood as indicating or implying relative importance or implying the number of technical features indicated. Thus, the features defined as “first”, “second”, “third”, and “fourth” may comprise at least one of these features explicitly or implicitly. In the description of this application, “a plurality of” means two or more, unless otherwise explicitly and specifically defined.

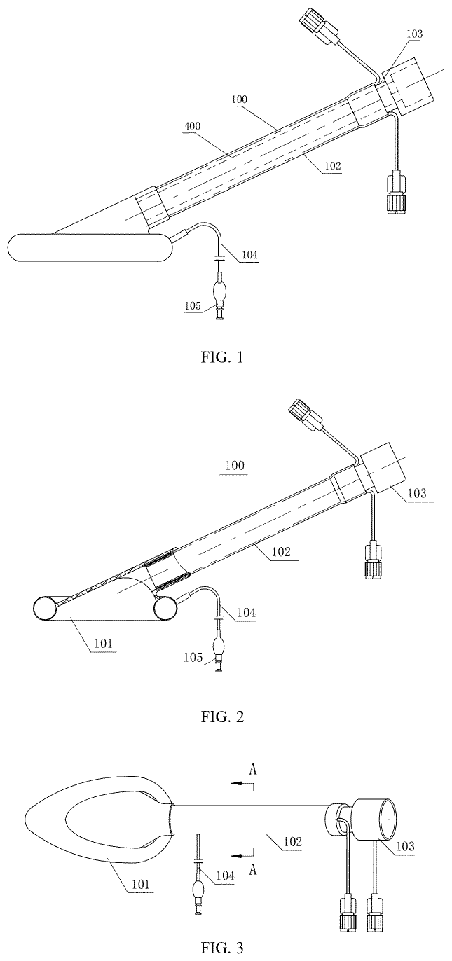

Referring to FIGS. 1 to 4a, the airway management system provided in the embodiment of this invention comprising a laryngeal mask component 100 and an inner tube component 400, the inner tube component 400 comprises an inner tube body 401, and a third ventilation cavity 403 is arranged inside the inner tube body 401; where the laryngeal mask component 100 comprises a mask body 101, a laryngeal mask airway tube 102 connected to one end of the mask body 101, a first ventilation cavity 106 is arranged inside the laryngeal mask airway tube 102, and the first ventilation cavity 106 is configured for inserting the inner tube component 400, after inserting the inner tube component 400, between the first ventilation cavity 106 and the third ventilation cavity 402, one forms an inhalation cavity and another forms an exhalation cavity;

The patient end of the inner tube component 400 is configured to be positioned within the mask body 101, and aligned with the patient end of the laryngeal mask airway tube 102; The patient end of the inner tube component 400 can also be configured to enter the trachea of the patient through the glottis of the patient.

By inserting the inner tube component 400 into the laryngeal mask component 100, the inner tube connector 403 and the laryngeal mask airway connector 103 jointly connect to the patient end of the breathing circuit connector 310, where the breathing circuit connector 310 is connected to an anesthesia machine or a ventilator through a breathing circuit, which can eliminate apparatus dead space and avoid that the patient inhales his/her own exhaled exhaust gas.

The apparatus dead space and some of the anatomic dead space are eliminated, which has clinical significance for patients who require low tidal volume and low-driving pressure ventilation. The patients who require low tidal volume and low-driving pressure ventilation comprise but are not limited to: 1 patients undergoing pediatric surgery; 2 patients undergoing laparoscopic surgery that require carbon dioxide filling; 3 patients undergoing thoracic surgery; 4 patients suffering from pulmonary diseases and undergoing general anesthesia; 5 patients undergoing massive transfusion and infusion surgery; 6 patients undergoing prolonged surgery; 7 patients with chronic obstructive pulmonary disease (COPD) or acute respiratory distress syndrome (ARDS); and 8 patients with severe pneumonia who need invasive ventilation treatment.

For ARDS patients, this application can achieve a good therapeutic effect in the supine position, and will achieve a better therapeutic effect if combined with the prone position ventilation treatment.

Different specifications of laryngeal mask components 100 or endotracheal tube components 400 can be used in combination with different specifications of inner tube components to meet the needs of patients with different glottic areas and different ventilator parameter settings.

Referring to FIGS. 1 to 4a, optionally, between the ventilation cavity formed after the first ventilation cavity 106 inserted into the inner tube component 400 and the third ventilation cavity 402, one forms an inhalation cavity and another forms an exhalation cavity; or

between the part of the first ventilation cavity 106 inserted into the inner tube component 400 and the third ventilation cavity 402, one forms an inhalation cavity and another forms an exhalation cavity.

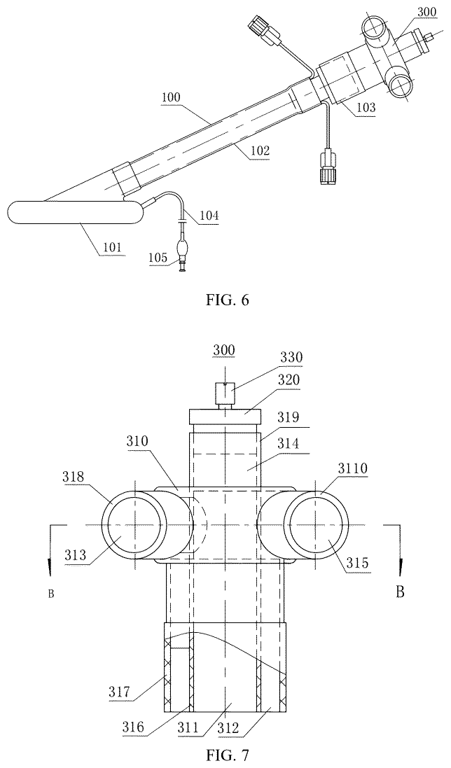

Referring to FIGS. 1 to 11, optionally, the laryngeal mask component 100 further comprises a laryngeal mask airway connector 103, where the other end of the laryngeal mask airway tube 102 is connected to the laryngeal mask airway connector 103; The inner tube component 400 also comprises an inner tube connector 403 connected to the inner tube body 401; The airway management system also comprises a breathing circuit connector component 300; the breathing circuit connector component 300 comprises a breathing circuit connector 310; one end of the breathing circuit connector 310 is connected to the laryngeal mask airway connector 103, or connected to the laryngeal mask airway connector 103 and the inner tube connector 403, and the other end of the breathing circuit connector 310 is configured to connect to an anesthesia machine or a ventilator through a breathing circuit;

The breathing circuit connector 310 comprises a patient end of the breathing circuit connector 310; the patient end of the breathing circuit connector 310 comprises a patient end inner connector 316 and a patient end outer connector 317, wherein the patient end outer connector 317 is configured to connect to the laryngeal mask airway connector 103, the patient end inner connector 316 is configured to connect to the inner tube connector 403.

The laryngeal mask airway connector 103 is connected to the patient end of the breathing circuit connector 310, and at this time, the breathing circuit connector top end cover 320 is placed on the machine end first inner connector 318; By inserting a machine end second inner connector 319 into the inner tube body 401, the inner tube connector of 403 and the machine end outer connector 3110 can be connected to the patient end of the breathing circuit respectively, where the machine end of the breathing circuit is connected to an anesthesia machine or a ventilator; as the inner tube body 401 is gradually inserted, the apparatus dead space in the airway management system gradually decrease; when the patient end of the inner tube component 400 is located in the mask body 101 of the laryngeal mask component 100, and is aligned with the patient end of the laryngeal mask airway tube 102, the apparatus dead space can be eliminated; at this time, the patient end of the inner tube component 400 passes through the glottis of the patient and enters the trachea of the patient, thereby eliminating all the apparatus dead space and some of the anatomic dead space. This method allows for the dead space volume of the airway management system to be continuously adjustable from being greater than that of the existing product to eliminating all the apparatus dead space plus some of the anatomic dead space, and is suitable for treating patients with hyperventilation.

The laryngeal mask airway tube 102 and the laryngeal mask airway connector 103 have internal diameters larger than that of the existing product, which can reduce the airway resistance, implant a diagnosis and treatment device with a larger external diameter and insert an endotracheal tube with a larger external diameter via the laryngeal mask airway tube 102 and the laryngeal mask airway connector 103. A sampling site of the laryngeal mask component 100 is close to the patient end of the catheter, which can make the sampling site close to the alveoli to the greatest extent, such that the sampling analysis is more sensitive and accurate, and a sampling analysis numerical value and an arterial blood gas analysis numerical value are more approximate and have good correlation.

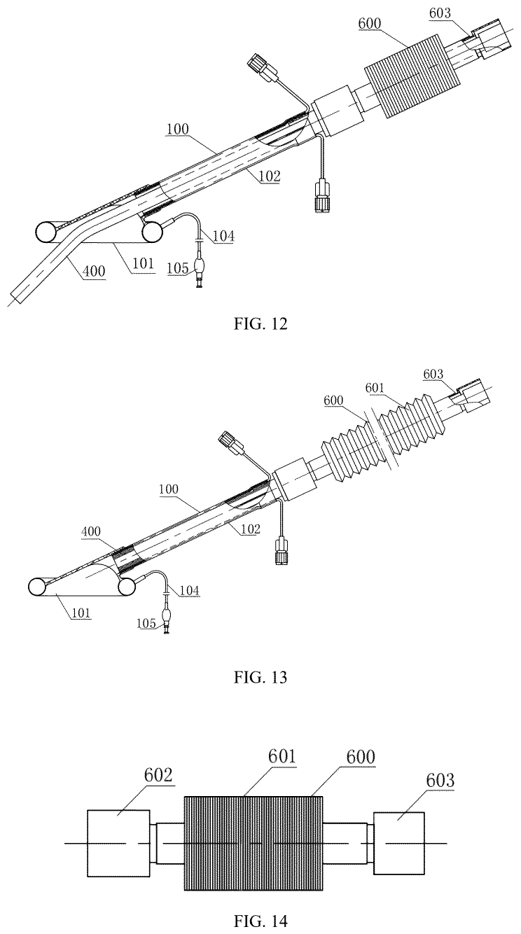

Referring to FIGS. 12 to 16, optionally, the airway management system further comprises a telescopic tube component 600, the telescopic tube component 600 comprises a telescopic tube patient end connector 602, a telescopic tube machine end connector 603, and a telescopic tube body 601, one end of the telescopic tube body 601 is connected to the telescopic tube patient end connector 602, the other end of the telescopic tube body 601 is connected to the telescopic tube machine end connector 603; the telescopic tube patient end 602 is configured to connect the laryngeal mask airway connector 103; the inner tube component 400 is configured to be inserted via the laryngeal mask airway tube 102, the laryngeal mask airway connector 103 and the telescopic tube component 600; the telescopic tube machine end connector 603 and the inner tube connector 403 are configured to connect to the patient end of the breathing circuit connector 310;

When the telescopic tube component 600 is configured to fully extend to its completely open state, the patient end of the inner tube component 400 reaches the trachea of the patient; when the telescopic tube component 600 gradually contracts, the patient end of the inner tube component 400 moves towards the lungs of the patient, passes through the glottis of the patient, and enters the trachea of the patient.

The laryngeal mask airway connector 103 can be connected to the patient end of the telescopic tube component 600; when the telescopic tube component 600 is fully extended to its completely open state, the inner tube component 400 can be inserted via the laryngeal mask component 100 and the telescopic tube component 600; the patient end of the inner tube component 400 is located on the glottis of the patient, and in the mask body 101 of the laryngeal mask component 100, and is aligned with the patient end of the laryngeal mask airway tube 102, the inner tube connector 403 and the telescopic tube machine end connector 603 are jointly connected to the patient end of the breathing circuit connector 310, and the breathing circuit connector 310 is connected to the anesthesia machine or the ventilator through the breathing circuit, which can eliminate apparatus dead space, avoid that a patient inhales his/her own exhaled exhaust gas from the laryngeal mask component 100, and can improve the sensitivity and accuracy of gas sampling analysis in the airway of the patient. As the telescopic tube component 600 gradually contracts, the patient end of the inner tube component 400 moves towards the lungs of the patient, passes through the glottis of the patient, and enters the trachea; when the telescopic tube component 600 contracts until it is in its original state, the patient end of the inner tube component 400 can be made reach the trachea of the patient, thereby eliminating all the apparatus dead space and some of anatomic dead space; and the inner tube connector 403 and the telescopic tube machine end connector 603 are jointly connected to the patient end of the breathing circuit connector 310, and the breathing circuit connector 310 is connected to the anesthesia machine or the ventilator through the breathing circuit.

After the inner tube component 400 is inserted via the laryngeal mask component 100 and telescopic tube component 600, the patient end of the inner tube component 400 can reach the trachea of the patient. Compared to an existing laryngeal mask product, a third ventilation cavity 402 of the inner tube component 400 can be selected as an inhalation channel, and a gap between the inner tube body 401 and the trachea, the glottis, and the laryngeal mask airway tube 102 may serve as an exhalation channel, which guarantees smooth respiration, reduces airway pressure, and lowers the risk of accidental aspiration. Due to the ventilation and positioning function of the inner tube component 400, the risk of airway obstruction is eliminated, and the stability of an artificial airway is increased. Compared to an existing endotracheal tube product, the risk of the mucosa of a tracheal lining is compressed after the cuff is inflated to cause mucosal ischemic necrosis is eliminated.

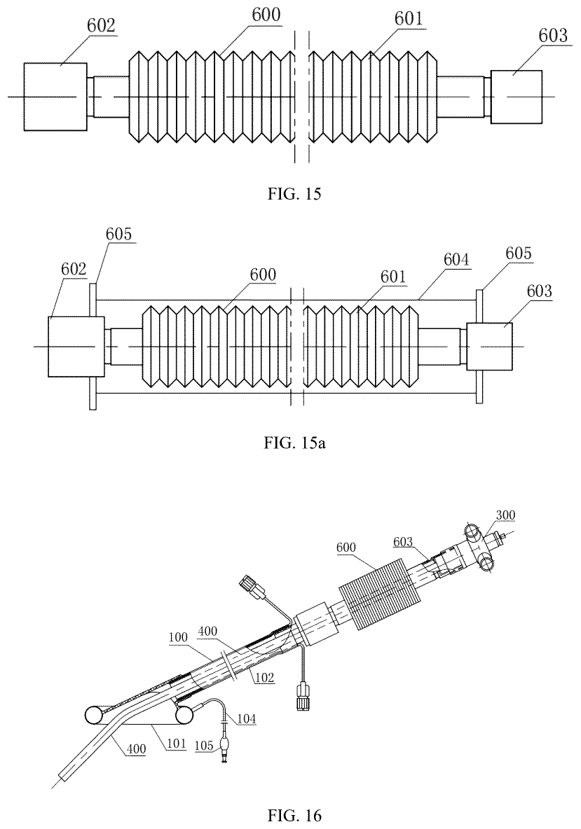

Referring to FIG. 15a, optionally, the telescopic tube component 600 further comprises: annular protrusions 605 and a stopper 604; the annular protrusions 605 are provided on outer side walls of the telescopic tube patient end connector 602 and the telescopic tube machine end connector 603, respectively; the stopper 604 is configured to limit the telescopic tube component 600; and when the telescopic tube component 600 is fully extended to its completely open state, the stopper 604 is located between the annular protrusions 605.

The laryngeal mask airway connector 103 can be connected to the patient end of the telescopic tube component 600; when the telescopic tube component 600 is fully extended to its completely open state, an optional way is to place the stopper 604 between the annular protrusions 605; the inner tube component 400 can be inserted via the laryngeal mask component 100 and the telescopic tube component 600; the patient end of the inner tube component 400 is located on the glottis of the patient, and in the mask body 101 of the laryngeal mask component 100, and is aligned with the patient end of the laryngeal mask airway tube 102, the inner tube connector 403 and the telescopic tube machine end connector 603 are jointly connected to the patient end of the breathing circuit connector 310, and the breathing circuit connector 310 is connected to the anesthesia machine or the ventilator through the breathing circuit, which can eliminate apparatus dead space, avoid that a patient inhales his/her own exhaled exhaust gas, and can improve the sensitivity and accuracy of gas sampling analysis in the airway of the patient.

The annular protrusions 605 of the telescopic tube component 600 are provided on the outer side walls of the telescopic tube patient end connector 602 and the telescopic tube machine end connector 603, respectively; the stopper 604 is configured to limit the telescopic tube component 600; and when the telescopic tube component 600 is fully extended to its completely open state, the stopper 604 is located between the annular protrusions 605, so that the telescopic tube component 600 cannot contract. This avoids that the patient end of the inner tube component 400 injures the glottis and trachea of the patient due to the fact that the telescopic tube component 600 contracts. After the stopper 604 is removed, as the telescopic tube component 600 gradually contracts, the patient end of the inner tube component 400 moves towards the lungs of the patient, passes through the glottis of the patient, and enters the trachea; when the telescopic tube component 600 contracts until it is in its original state, the patient end of the inner tube component 400 can be made reach the trachea of the patient, thereby eliminating all the apparatus dead space and some of anatomic dead space; and the inner tube connector 403 and the telescopic tube machine end connector 603 are jointly connected to the patient end of the breathing circuit connector 310, and the breathing circuit connector 310 is connected to the anesthesia machine or the ventilator through the breathing circuit.

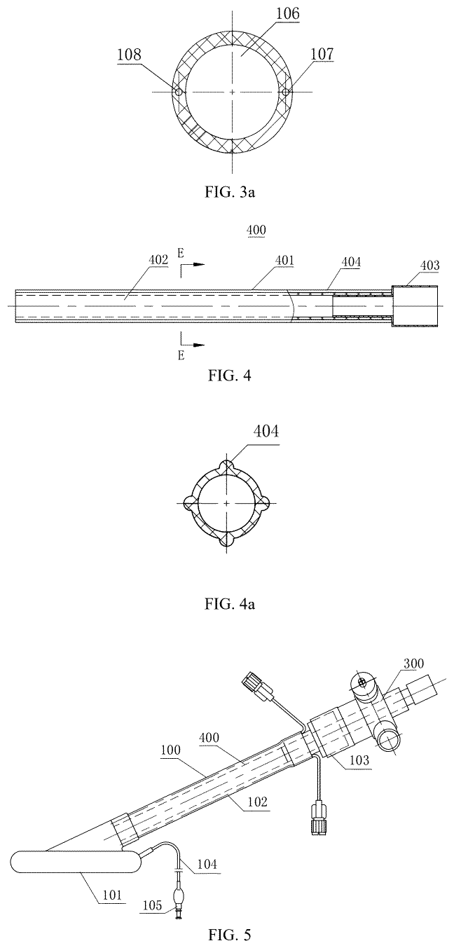

Referring to FIG. 3a, optionally, a first gas sampling cavity 108, and a first throat aspiration cavity 107 are further provided in the laryngeal mask airway tube 102.

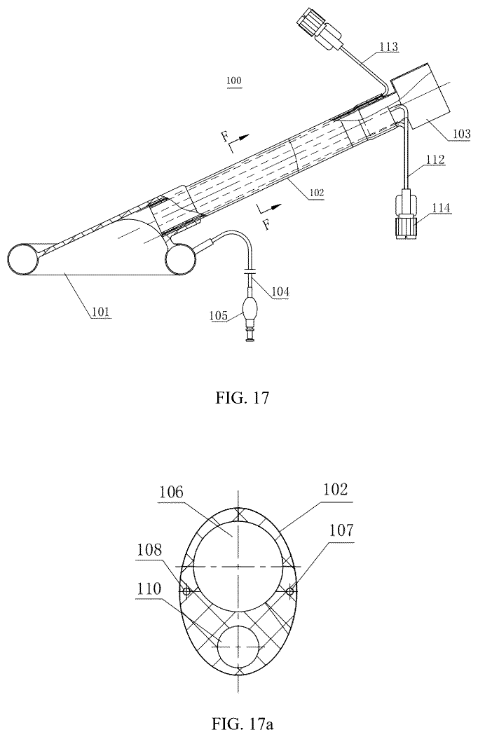

Referring to FIGS. 17 and 17a, optionally, a drainage cavity 110 is further provided in the laryngeal mask airway tube 102.

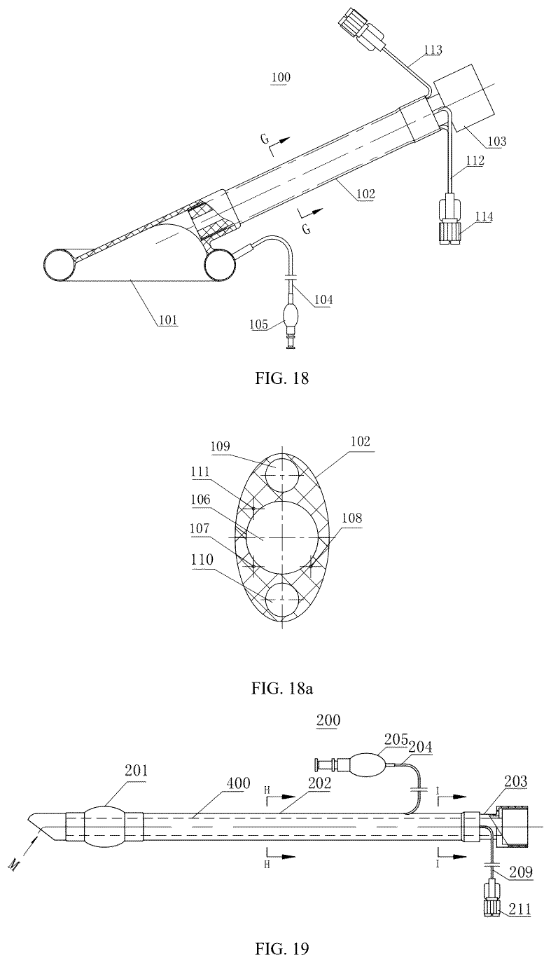

Referring to FIGS. 18 and 18a, optionally, a video component cavity 109 is further provided in the laryngeal mask airway tube 102.

Referring to FIGS. 18 and 18a, optionally, a standby reserved cavity 111 is further provided in the laryngeal mask airway tube 102.

Referring to FIGS. 2 and 3a, optionally, the patient end of the first gas sampling cavity 108 and the patient end of the first throat aspiration cavity 107 are close to the patient end of the laryngeal mask airway tube 102, the first gas sampling cavity 108 inside the laryngeal mask airway tube 102 and the first throat aspiration cavity 107 inside the laryngeal mask airway tube 102 are fixedly mated.

The patient end of the first gas sampling cavity 108 of the laryngeal mask component 100 and the first gas sampling cavity 108 are located on the ventral side of the patient. The patient end of the first gas sampling cavity 108 is close to the patient end of the catheter, which can improve the sensitivity and accuracy of gas sampling analysis in the airway of the patient. The patient end of the first throat aspiration cavity 107 of the laryngeal mask component 100 and the throat aspiration cavity are located on the dorsal side of the patient; the patient end of the first throat aspiration cavity 107 is close to the patient end of the laryngeal mask airway tube 102; and the first throat aspiration cavity 107 can aspirate secretions near the glottis of the patient and or may serve as a standby gas sampling cavity. The patient end of the first gas sampling cavity 108, the first gas sampling cavity 108 and the first throat aspiration cavity 107 are fixedly mated, and are symmetrically provided, which guarantees the continuity and reliability of gas sampling analysis.

The first gas sampling cavity 108 of the laryngeal mask component 100 can be provided in the tube of the laryngeal mask airway tube 102, or may be independent outside the laryngeal mask airway tube 102. The first throat aspiration cavity 107 and the first gas sampling cavity 108 are symmetrically positioned and have identical structures. The patient end of the first throat aspiration cavity 107 and the patient end of the first gas sampling chamber 108 are close to the patient end of the catheter, symmetrically positioned, and have identical structures. The first throat aspiration cavity 107 can be used to suction pharyngeal secretions, and can also serve as a standby sampling cavity when the gas sampling cavity is blocked, ensuring the continuity and reliability of gas sampling analysis.

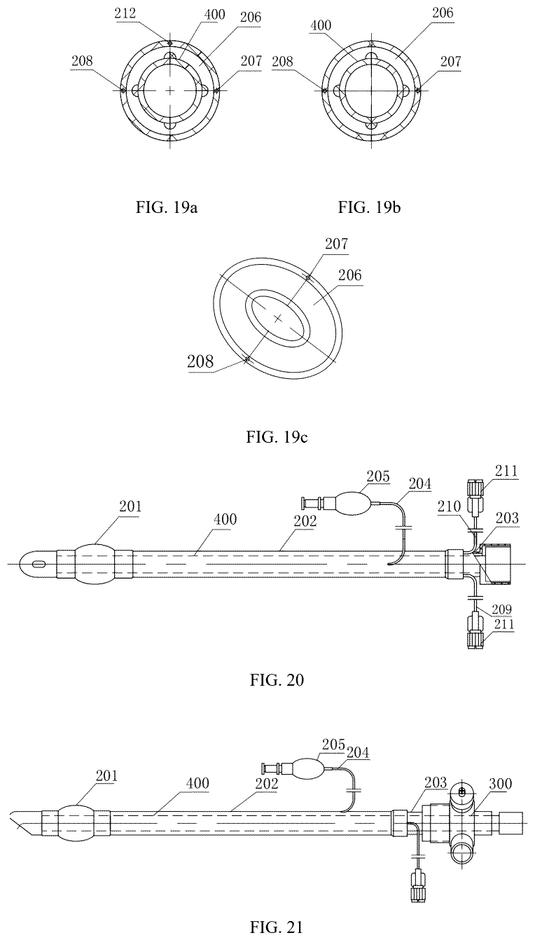

As shown in FIG. 19-FIG. 21, another embodiment of the invention provides an airway management system, comprises: an endotracheal tube component 200 and an inner tube component 400, wherein the inner tube component 400 comprises an inner tube body 401, a third ventilation cavity 402 is arranged inside the inner tube body 410. The endotracheal tube component 200 comprises an endotracheal tube 202, a second ventilation cavity 206 is arranged in the endotracheal tube 202. The second ventilation cavity 206 is configured for inserting the inner tube component 400; after inserting the inner tube component 400, between the second ventilation cavity 206 and the third ventilation cavity 402, one forms an inhalation cavity and another forms an exhalation cavity. The endotracheal tube 202 has the same internal diameter. The patient end of the inner tube component 400 is configured to be align with the patient end of the endotracheal tube 202. The patient end of the inner tube component 400 is also configured to enter into the trachea of the patient through the patient end of the endotracheal tube 202.

By inserting the endotracheal tube component 200 into the inner tube component 400, the inner tube connector 403 and the endotracheal tube connector 203 jointly connect to the patient end of the breathing circuit connector 310, where the breathing circuit connector 310 is connected to an anesthesia machine or a ventilator through a breathing circuit, which can eliminate apparatus dead space and avoid that the patient inhales his/her own exhaled exhaust gas.

The endotracheal tube connector 203 of the endotracheal tube component 200 can be connected to the patient end of the breathing circuit connector 310, and at this time, the breathing circuit connector top end cover 320 is placed on the machine end first inner connector 318 of the breathing circuit connector 310. By inserting the machine end second inner connector 319 into the inner tube body 401, the inner tube connector 403 of the inner tube component and the machine end outer connector 3110 of the breathing circuit connector 310 are each connected to the patient end of the breathing circuit, and the machine end of the breathing circuit is connected to the anesthesia machine or the ventilator. The endotracheal tube connector 203 of the endotracheal tube component 200 in which internal diameters are equal is connected to the patient end of the breathing circuit connector 310, and after the machine end second inner connector 319 is inserted into the inner tube body 401; as the inner tube body 400 is gradually inserted, the apparatus dead space in the airway management system gradually decrease, when the patient end of the inner tube component 400 and the patient end of the endotracheal tube 202 are aligned, apparatus dead space is eliminated; the patient end of the inner tube component 400 is further inserted and enters the trachea of the patient, thereby eliminating all the apparatus dead space and some of the anatomic dead space.

Endotracheal tube components 200 of different specifications can be used in combination with inner tube components 400 of different specifications to meet the needs of patients with different glottal areas and different ventilator parameter settings.

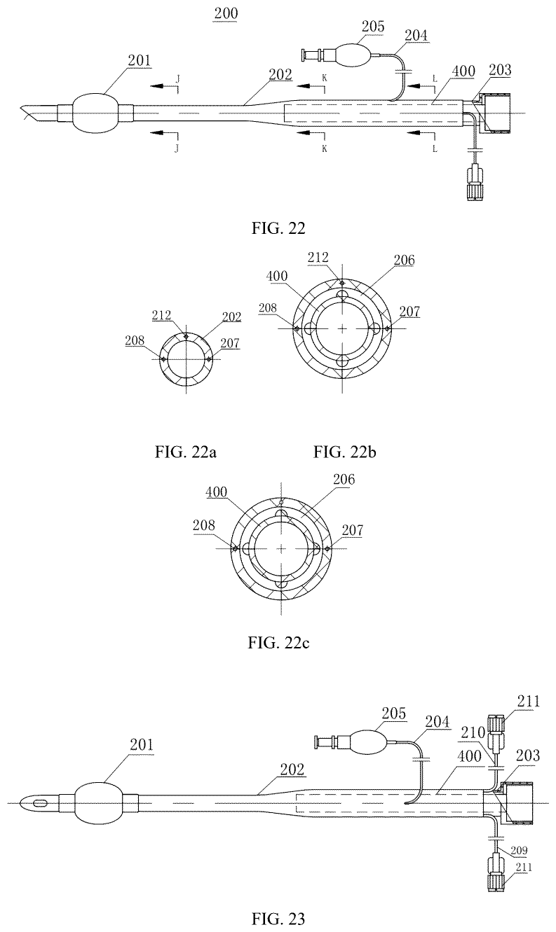

As shown in FIG. 22-FIG. 24, another embodiment of this invention provides an airway management system, comprises an endotracheal tube component 200 and an inner tube component 400, wherein the inner tube component 200 comprises an inner tube body 202, a third ventilation cavity 206 is arranged inside the inner tube body 202. The second ventilation cavity 206 is configured for inserting the inner tube component 400; after inserting the inner tube component 400, between the second ventilation cavity 206 and the third ventilation cavity 402, one forms an inhalation cavity and another forms an exhalation cavity. The internal diameter of the endotracheal tube 202 is configured such that the internal diameter of the segment located below the glottis of a patient when in use is smaller than that of the segment located above the glottis of the patient when in use. The patient end of the inner tube component 400 is configured to be located at the patient end of the large diameter portion of the endotracheal tube 202 of the unequal diameter structure.

By inserting the endotracheal tube 202 into the inner tube component 400, the inner tube connector 403 and the endotracheal tube connector 203 are jointly connected to the patient end of the breathing circuit connector 310. The breathing circuit connector 310 is connected to the anesthesia machine or the ventilator through the breathing circuit, which can reduce or even eliminate apparatus dead space and further avoid that a patient inhales his/her own exhaled exhaust gas.

The endotracheal tube connector 203 of the endotracheal tube component 200 can be connected to the patient end of the breathing circuit connector 310, and at this time, the breathing circuit connector top end cover 320 is placed on the machine end first inner connector 318 of the breathing circuit connector 310. By inserting the machine end second inner connector 319 into the inner tube body 401, the inner tube connector 403 of the inner tube component and the machine end outer connector 3110 of the breathing circuit connector 310 are each connected to the patient end of the breathing circuit, and the machine end of the breathing circuit is connected to the anesthesia machine or the ventilator. The endotracheal tube connector 203 of the endotracheal tube component 200 in which internal diameters are unequal is connected to the patient end of the breathing circuit connector 310, and the machine end second inner connector 319 is inserted into the inner tube body 401; as the inner tube body 400 is gradually inserted, the apparatus dead space in the airway management system gradually decrease, when the patient end of the inner tube component 400 is located at the patient end of the large diameter portion of the endotracheal tube 202 of the unequal diameter structure, thereby eliminating some of the anatomic dead space.

Endotracheal tube components 200 of different specifications can be used in combination with inner tube components 400 of different specifications to meet the needs of patients with different glottal areas and different ventilator parameter settings.

The internal diameter of the endotracheal tube 202 in this invention could be configured such that the internal diameter of the segment located below the glottis of a patient when in use is smaller than that of the segment located above the glottis of the patient when in use. This can avoid injure to the patient's glottis and trachea, improve patient comfort and reduce complications.

As shown in FIG. 19-FIG. 21, optionally, between the ventilation cavity formed after the second ventilation cavity 206 inserted into the inner tube component 400 and the third ventilation cavity 402, one forms an inhalation cavity and another forms an exhalation cavity; or

between the part of the second ventilation cavity 206 inserted into the inner tube component 400 and the third ventilation cavity 402, one forms an inhalation cavity and another forms an exhalation cavity.

As shown in FIG. 19-FIG. 24, optionally, the endotracheal tube component 200 further comprises an endotracheal tube connector 203. The inner tube component 400 comprises an inner tube connector 403 connected to the inner tube body 401. The airway management system further comprises a breathing circuit connector component 300, the breathing circuit connector component 300 comprises a breathing circuit connector 310, one end of the breathing circuit connector 310 is connected to the endotracheal tube connector 203 or to the endotracheal tube connector 203 and the inner tube connector 403, the other end of the breathing circuit connector 310 is configured to be connected to the anesthesia machine or ventilator via breathing circuit;

The breathing circuit connector 310 comprises a patient end and a machine end of the breathing circuit connector 310. The patient end of the breathing circuit connector 310 comprises a patient end inner connector 316 and a patient end outer connector 317. The patient end outer connector 317 is configured to be connected to the endotracheal tube connector 203, the patient end inner connector 316 is configured to be connected to the inner tube connector 403.

The endotracheal tube 202 and the endotracheal tube connector 203 of this invention have internal diameters larger than that of the existing product, a diagnosis and treatment device with a larger external diameter can be implanted. A sampling site of the endotracheal tube component 200 is close to the patient end of the catheter, which can make the sampling site close to the alveoli to the greatest extent, such that the sampling analysis is more sensitive and accurate, and a sampling analysis numerical value and an arterial blood gas analysis numerical value are more approximate and have good correlation.

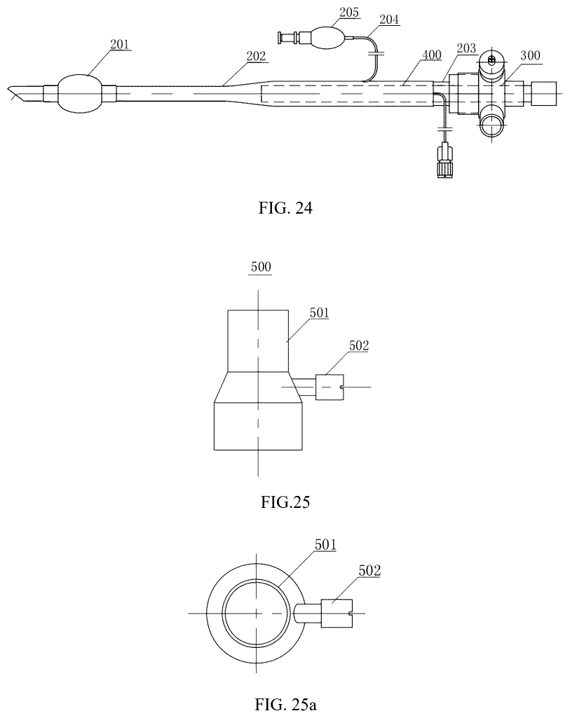

As shown in FIG. 7-FIG. 11, and FIG. 21, FIG. 24, optionally, the machine end of the breathing circuit connector 310 comprises a machine end first inner connector 318, a machine end second inner connector 319, and a machine end outer connector 3110. The machine end of the breathing circuit connector 310 is provided with three channels, the three channels being a machine end first inner channel 313, a machine end second inner channel 314, and a machine end outer channel 315. The patient end of the breathing circuit connector 310 comprises a patient end inner connector 311, and a patient end outer connector 312. The machine end first inner channel 313 and the machine end second inner channel 314 is connected to the patient end inner connector 311, the machine end outer channel 315 is connected to the patient end outer channel 312. The patient end inner channel 311 and the patient end outer channel 312 are of a coaxial inner and outer dual-channel structure. The machine end first inner connector 318 and the machine end outer connector 3110 or the machine end second inner connector 319 and the machine end outer connector 3110 are connected to the anesthesia machine or the ventilator through the breathing circuit, respectively.

After the laryngeal mask airway connector 103 or the endotracheal tube connector 203 is connected to the patient end of the breathing circuit connector component 300, the breathing circuit connector component 300 can rotated in any direction within a circular range, facilitating the insertion and removal of the laryngeal mask airway connector 103 of the laryngeal mask component 100 or the endotracheal tube connector 203 of the endotracheal tube component 200 with the patient end of the breathing circuit connector component 300, and facilitating the placement of the breathing circuit.

The breathing circuit connector component 300 employs a dual-channel design at the patient end and a three-channel design at the machine end, which can guarantee that the laryngeal mask component 100 or the endotracheal tube component 200 is implanted into the patient. After the laryngeal mask airway connector 103 of the laryngeal mask component 100 or the endotracheal tube connector 203 of the laryngeal mask component 200 is connected to the patient end of the breathing circuit connector component 300, the inner tube component 400 is implanted under the guidance of a fiberoptic bronchoscope via the machine end second inner connector 319 of the breathing circuit connector 310 without interrupting low dead space volume ventilation. The patient end of the first Luer taper 330 of the breathing circuit connector component 300 of this invention can be connected to the gas-sampling line, and the gas-sampling line can collect gas inside the laryngeal mask airway tube 102, the endotracheal tube 202, on the glottis or inside the trachea for detection.

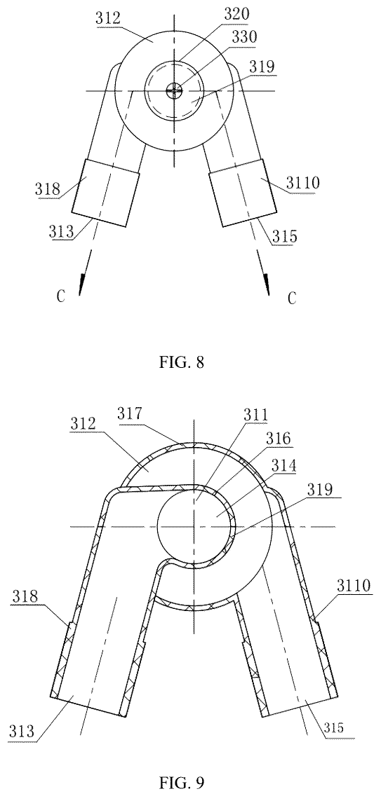

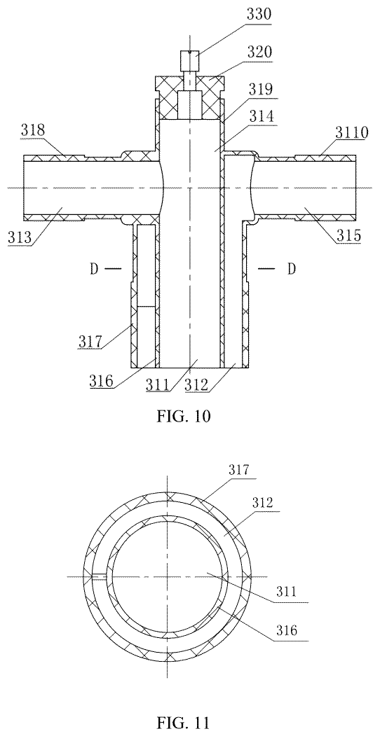

As shown in FIG. 7, FIG. 8, and FIG. 10, optionally, the breathing circuit connector component 300 further comprises a breathing circuit connector top end cover 320. The openable breathing circuit connector top end cover 320 is provided at the top end of the machine end second inner connector 319. The breathing circuit connector top end cover 320 is configured to be connected to the machine end first inner connector 318.

The laryngeal mask airway connector 103 of the laryngeal mask component 100 is connected to the patient end of the breathing circuit connector 310, or the endotracheal tube connector 203 of the endotracheal tube component 200 in which internal diameters are equal is connected to the patient end of the breathing circuit connector 310, or the endotracheal tube connector 203 of the endotracheal tube component 200 in which internal diameters are unequal is connected to the patient end of the breathing circuit connector 310, and at this time, the breathing circuit connector top end cover 320 is placed on the machine end first inner connector 318 of the breathing circuit connector 310.

As shown in FIG. 7, FIG. 8, and FIG. 10, optionally, the breathing circuit connector component 300 further comprises a first Luer taper 330; and the middle of the breathing circuit connector top end cover 320 is connected to the first Luer taper 330.

As shown in FIG. 25 and FIG. 25a, optionally, the airway management system further comprises a connecting tube 500, and the connecting tube 500 is of an unequal diameter structure. The connecting tube 500 comprises a connecting tube body 501. When clinical anesthesia induction is performed, one end of the connecting tube body 500 is configured to be connected to the patient end of the breathing circuit connector 310, and the other end of the connecting tube body 501 is configured to be connected to the anesthetic mask.

The connecting tube 500 of this application is designed as the unequal diameter structure, so that one end is connected to the patient end of the breathing circuit connector 310 and the other end is connected to the anesthesia mask or a common endotracheal tube.

As shown in FIG. 25 and FIG. 25a, optionally, the connecting tube 500 further comprises a second Luer taper 502. The second Luer taper 502 is configured to be connected to the gas-sampling line of the monitor.

The connecting tube 500 of this application is designed as the unequal diameter structure, and the second Luer taper 502 of the connecting tube 500 can serve as a gas sampling site during anesthesia induction and anesthesia maintenance. The patient end of the connecting tube 500 can be connected to an existing mask, an existing laryngeal mask, the existing endotracheal tube, the laryngeal mask component 100 of this application, the endotracheal tube component 200 of this application, the laryngeal mask component 100 and the inner tube component 400 of this application, and the endotracheal tube component 200 and the inner tube component 400 of this application, and the machine end of the connecting tube 500 is connected to the patient end of the breathing circuit connector component 300. After connection, the apparatus dead space can be enlarged, which is suitable for the induction stage of general anesthesia and the treatment of patients with hyperventilation.

As shown in FIG. 4 and FIG. 4a, optionally, an outer wall of the inner tube body 401 is provided with a plurality of protrusions 404; the protrusion 404 is configured for positioning between the inner tube body 401 and the first ventilation cavity 106 or the second ventilation cavity 206.

There are several protrusions 404 symmetrically designed in the axial direction on the outer wall of the inner tube body 401 in this invention, and after inserting into the ventilation cavity of the laryngeal mask airway tube 102 or endotracheal tube 202, the inner tube body 401 can be kept at the axis center position of the ventilation cavity into which the inner tube body is inserted under the support of the protrusions 404, which guarantees that the lumen of the ventilation cavity between the inner tube body 401 and the laryngeal mask airway tube 102 or the endotracheal tube 202 is unimpeded. The inner tube component 400 is of a detachable structure, and inner tube components 400 of different internal diameters, external diameters, and lengths can be selected according to the anatomical characteristics of the throat and trachea of the patient and clinical use needs.

As shown in FIG. 19a-FIG. 19c and FIG. 22a-FIG. 22c, optionally, a second throat aspiration cavity 207, a second gas sampling cavity 208, and a cuff inflation cavity 212 are provided in the endotracheal tube 202.

As shown in FIG. 19a-FIG. 24, optionally, the patient end of the second gas sampling cavity 208 and the patient end of the second throat aspiration cavity 207 are close to the patient end of the endotracheal tube 202. The second gas sampling cavity 208 and the second throat aspiration cavity 207 are fixedly mated.

The patient end of the second gas sampling cavity 208 of the endotracheal tube component 200 and the gas sampling cavity are located on the ventral side of the patient. The patient end of the second gas sampling cavity 208 is close to the patient end of the catheter, which can improve the sensitivity and accuracy of gas sampling analysis in the airway of the patient. The patient end of the second throat aspiration cavity 207 of the endotracheal tube component 200 and the throat aspiration cavity are located on the dorsal side of the patient; the patient end of the second throat aspiration cavity 207 is close to the patient end of the catheter; and the second throat aspiration cavity 207 can aspirate secretions near the glottis of the patient and or may serve as a standby gas sampling cavity. The patient end of the second gas sampling cavity 208, the patient end of the second gas sampling cavity 208 and of the second throat aspiration cavity 207, the second throat aspiration cavity 207 are fixedly mated, and are symmetrically provided, which guarantees the continuity and reliability of gas sampling analysis.

The second gas sampling cavity 208 of the endotracheal tube component 200 of this invention can be provided in the tube of the endotracheal tube 202, or may be independent outside the endotracheal tube 202, thus facilitating the design, research and development, and production of products.

### Embodiment 1

Referring to FIGS. 1 to 16a and FIG. 25, FIG. 25a, an airway management system as shown comprises a laryngeal mask component 100, a breathing circuit connector component 300, an inner tube component 400, a connecting tube 500, and a telescopic tube component 600.

As shown in FIGS. 2 to 3a, the laryngeal mask component 100 comprises a mask body 101, a laryngeal mask airway tube 102, a laryngeal mask airway connector 103, a laryngeal mask inflation tube 104, and a laryngeal mask inflation valve 105. One end of the laryngeal mask airway tube 102 is communicated with the mask body 101, and the other end of the laryngeal mask airway tube is communicated with the laryngeal mask airway connector 103. The laryngeal mask inflation tube 104 is provided on the exterior of the laryngeal mask airway tube 102. One end of the laryngeal mask inflation tube 104 is communicated with the mask body 101 to inflate the mask body, and the other end of the laryngeal mask inflation tube 104 is connected to the laryngeal mask inflation valve 105. As shown in FIG. 3a, three cavities are provided in the laryngeal mask airway tube 102, where the three cavities are a first ventilation cavity 106, a first gas sampling cavity 108, and a first throat aspiration cavity 107, respectively, where the first ventilation cavity 106 is located on a middle portion of the laryngeal mask airway tube 102 to facilitate insertion of the inner tube component 4. The first gas sampling cavity 108 and the first throat aspiration cavity 107 are each placed in the laryngeal mask airway tube 102, and a patient end of the first gas sampling cavity 108 and a patient end of the first throat aspiration cavity 107 are close to a patient end of the laryngeal mask airway tube 102. The first gas sampling cavity 108 is communicated with a first gas-sampling line 112, the first throat aspiration cavity 107 is communicated with a first throat aspiration tube 113, and the other end of the first gas-sampling line 112 and the other end of the first throat aspiration tube 113 are each connected to a third Luer taper 114. The third Luer taper 114 is connected to a monitor for analyzing the composition and concentration of the inhaled or exhaled gas of a patient. The first throat aspiration tube 113 may serve as an aspiration channel for secretions from the throat of the patient or may serve as a standby gas-sampling line.

An internal diameter that can be employed for the laryngeal mask airway tube 102 ranges from 3.3 to 25 mm. In some embodiments, the internal diameter of the laryngeal mask airway tube 102 may be any integer between 4 mm and 25 mm, or may also be 3.5 mm, 4.5 mm, 5.5 mm, 6.5 mm, 7.5 mm, 8.5 mm, 9.5 mm, 10.5 mm, 11.5 mm, 12.5 mm, 13.5 mm, 14.5 mm, 15.5 mm, 16.5 mm, 17.5 mm, 18.5 mm, 19.5 mm, 20.5 mm, 21.5 mm, 22.5 mm, 23.5 mm, 24.5 mm and so on. An internal diameter that can be employed for the inner tube body 401 ranges from 2 to 12 mm. In some embodiments, the internal diameter of the inner tube body 401 may be any integer between 2 mm and 12 mm, or may also be 2.5 mm, 3.5 mm, 4.5 mm, 5.5 mm, 6.5 mm, 7.5 mm, 8.5 mm, 9.5 mm, 10.5 mm, 11.5 mm and so on.

In addition, the mask body 101 can adopt the following specifications: 1#, 1.5#, 2#, 2.5#, 3#, 4#, 5#, 6#, etc. These specifications and dimensions are common sizes of existing products on the market, which will not be elaborated here.

As shown in FIG. 7-FIG. 11, the breathing circuit connector component 300 comprises a breathing circuit connector 310, a breathing circuit connector top end cover 320, and a first Luer taper 330. The breathing circuit connector 310 comprises the patient end of the breathing circuit connector 310 and the machine end of the breathing circuit connector 310. The patient end of the breathing circuit connector 310 comprises a patient end inner connector 316 and a patient end outer connector 317. The machine end of the breathing circuit connector 310 comprises a machine end first inner connector 318, a machine end second inner connector 319, and a machine end outer connector 3110; The patient end is equipped with a patient end inner channel 311 and a patient end outer channel 312, where the patient end inner channel 311 and patient end outer channel 312 adopt a coaxial inner and outer dual-channel structure. The machine end of the breathing circuit connector 310 contains three channels, namely the machine end first inner channel 313 located within the machine end first inner connector 318, the machine end second inner channel 314 within the machine end second inner connector 319, and the machine end outer channel 315 within the machine end outer connector 3110; The machine end first inner channel 313, the machine end second inner channel 314, and the patient end inner channel 311 are connected. The machine end outer channel 315 and the patient end outer channel 312 are connected. An openable breathing circuit connector top end cover 320 is installed at the top of the machine end second inner connector 319. The first Luer taper 330 is connected to the center of the breathing circuit connector top end cover 320. During clinical use, the patient end outer connector 317 can connect to the laryngeal mask airway connector 103 or the telescopic tube patient end connector 602, while the patient end inner connector 316 connects to the inner tube connector 403. The machine end first inner connector 318 and the machine end outer connector 3110 connect to the breathing circuit, which connects to the anesthesia machine or ventilator. When the breathing circuit connector top end cover 320 is removed, devices such as a fiberoptic bronchoscope, closed suction tube, or bronchial occluder can be inserted through the machine end second inner channel 314; Alternatively, the machine end second inner connector 319 and the machine end outer connector 3110 can be connected to the breathing circuit. Remove the breathing circuit connector top end cover 320 and place it at the machine end first inner connector 318, with the breathing circuit connected to the anesthesia machine or ventilator.

As shown in FIG. 4-FIG. 4a, the inner tube component 400 comprises an inner tube body 401 and an inner tube connector 403, with a third ventilation cavity 402 set inside the inner tube body 401. According to clinical needs, the inner tube component 400, closed suction tube, or bronchial occluder, etc. can be inserted into the first ventilation cavity 106; After inserting the inner tube component 400, a closed suction tube or bronchial occluder can also be inserted into the inner tube body 401. The outer wall of the inner tube body 401 has multiple symmetrically arranged protrusions 404 along the axial direction, which are used for positioning the inner tube body 401 and the first ventilation cavity 106 inside the Laryngeal mask airway tube 102.

As shown in FIG. 25-FIG. 25a, the connecting tube 500 is of an unequal diameter structure. An equal diameter structure refers to a structure where the tube diameter (inner or external diameter) is completely identical or approximately identical throughout the connecting tube 500. The unequal diameter structure is opposite to the equal diameter structure. An unequal diameter structure refers to a structure where the tube diameter varies throughout the connecting tube 500, meaning the connecting tube 500 has different inner (outer) diameter dimensions/sizes. Additionally, the connecting tube 500 has the same wall thickness. The connecting tube 500 comprises a connecting tube body 501 and a second Luer taper 502. In clinical use, one end of the connecting tube body 501 connects to the patient end of the breathing circuit connector 310, while the other end connects to the anaesthetic mask. The second Luer taper 502 connects to the gas-sampling line of the monitor, for use during anesthesia induction; One end of the connecting tube 500 can also connect to the connector of a standard endotracheal tube, with the other end connecting to the patient end of the breathing circuit connector 310. The second Luer taper 502 connects to the gas-sampling line of the monitor, for use during intubation treatment via the laryngeal mask component 100.

As shown in FIG. 1 and FIGS. 12-16, the telescopic tube component 600 comprises a telescopic tube body 601, a telescopic tube patient end connector 602, and a telescopic tube machine end connector 603. The telescopic tube patient end connector 602 connects to the laryngeal mask airway connector 103, and the telescopic tube machine end connector 603 connects to the patient end of the breathing circuit connector 310. The laryngeal mask airway connector 103 can connect to the telescopic tube patient end connector 602. When the telescopic tube component 600 is fully extended to its completely open state, the inner tube component 400 can be inserted through the laryngeal mask component 100 and the telescopic tube component 600. The patient end of the inner tube component 400 is positioned above the patient's glottis, inside the mask body 101, and aligned with the patient end of the Laryngeal mask airway tube 102, which can eliminate the apparatus dead space. As the telescopic tube component 600 gradually contracts, the patient end of the inner tube component 400 moves toward the patient's lungs, passes through the patient's glottis, and enters the patient's trachea. When the telescopic tube component 600 contracts until it returns to its original state, the patient end of the inner tube component 400 reaches the patient's trachea, eliminating all apparatus dead space and part of the anatomic dead space.

During clinical implementation of anesthesia induction, the connecting tube 500 connects the patient end of the breathing circuit connector 310 to the anaesthetic mask, and the second Luer taper 502 connects to the gas-sampling line of the monitor. The anaesthetic mask covers the patient's nose and mouth, and the breathing circuit connector 310 connects to the anesthesia machine or ventilator through the breathing circuit.

In one embodiment, as shown in FIG. 15a, the telescopic tube component 600 of this invention may also comprise: annular protrusions 605 and a stopper 604. The annular protrusions 605 are respectively set on the outer sidewalls of the telescopic tube patient end connector 602 and the telescopic tube machine end connector 603. The stopper 604 is used to limit the telescopic tube component 600. When the telescopic tube component 600 is fully extended to its completely open state, the stopper 604 is positioned between the annular protrusions 605. During use, when the telescopic tube component 600 is fully extended to its completely open state, the stopper 604 is placed between the annular protrusions 605. The inner tube component 400 can be inserted through the laryngeal mask component 100 and the telescopic tube component 600. The patient end of the inner tube component 400 is positioned above the patient's glottis, inside the mask body 101, and aligned with the patient end of the Laryngeal mask airway tube 102. After removing the stopper 604, as the telescopic tube component 600 gradually contracts, the patient end of the inner tube component 400 moves toward the patient's lungs, passes through the patient's glottis, and enters the patient's trachea. The stopper 604 can be designed as a separable hollow cylindrical tube, as shown in FIG. 15a, where the portion of the stopper 604 located above the telescopic tube body 601 can be the separable part. The stopper 604 can be inserted or removed through the separable part of the stopper 604, allowing the telescopic tube component 600 to be positioned inside or outside the hollow cylindrical tube, thereby achieving the limiting or non-limiting function for the telescopic tube component 600.

The following will describe in detail the usage/working method of the airway management system in this application.

During anesthesia maintenance, after the patient is inserted with the Laryngeal mask component 100, connect the Laryngeal mask airway connector 103 to the patient end of the breathing circuit connector 310, and the breathing circuit connector 310 is connected to the anesthesia machine or ventilator through the breathing circuit. The third Luer taper 114 at the end of the first gas-sampling line 112 connects to the gas-sampling line of the monitor to analyze the composition and concentration of gases inhaled or exhaled by the patient. The machine end inner connector 318 and the machine end outer connector 3110 connect to the breathing circuit, or alternatively, the machine end second inner connector 319 and the machine end outer connector 3110 connect to the breathing circuit, which connects to the anesthesia machine or ventilator. The machine end first inner connector 318 and machine end outer connector 3110 connect to the breathing circuit, when the breathing circuit connects to the anesthesia machine or ventilator, a fiberoptic bronchoscope, closed suction tube, bronchial occluder, etc. can be inserted through the machine end second inner channel 314.

During anesthesia maintenance, after the patient is inserted with the laryngeal mask component 100, the inner tube component 400 can also be inserted through the Laryngeal mask airway tube 102 and laryngeal mask airway connector 103, with the laryngeal mask airway connector 103 and inner tube connector 403 jointly connecting to the patient end of the breathing circuit connector 310. The patient end outer connector 317 connects to the laryngeal mask airway connector 103, and the patient end inner connector 316 connects to the inner tube connector 403; The machine end first inner connector 318 and machine end outer connector 3110 connect to the breathing circuit, alternatively the machine end second inner connector 319 and machine end outer connector 3110 can connect to the breathing circuit, which connects to the anesthesia machine or ventilator, eliminating apparatus dead space.

During anesthesia maintenance, after the patient is inserted with the laryngeal mask component 100, the laryngeal mask component 100 is connected to the patient end of the breathing circuit connector component 300, then the breathing circuit connector top end cover 320 is removed and placed on the machine end inner connector 318. The inner tube body 401 is inserted through the machine end second inner connector 319, the inner tube connector 403 and the machine end outer connector 3110 are connected to the patient end of the breathing circuit respectively, while the machine end of the breathing circuit is connected to the anesthesia machine or ventilator. The laryngeal mask component 100 connects to the patient end of the breathing circuit connector 310, and after the inner tube body 401 is inserted from the machine end second inner connector 319, the inner tube connector 403 and the machine end outer connector 3110 respectively connect to the patient end of the breathing circuit, while the machine end of the breathing circuit connects to the anesthesia machine or ventilator. As the inner tube body 401 is gradually inserted, the apparatus dead space within the airway management system gradually decreases; when the patient end of the inner tube component 400 is positioned inside the mask body 101 and aligned with the patient end of the Laryngeal mask airway tube 102, the apparatus dead space is eliminated; The patient end of the inner tube component 400 can enter the trachea through the glottis, thereby eliminating all apparatus dead space and part of the anatomic dead space.

During anesthesia maintenance, after the patient is inserted with the laryngeal mask component 100, the laryngeal mask airway connector 103 can also be connected to the telescopic tube patient end connector 602. When the telescopic tube component 600 is fully extended to its completely open state, the inner tube component 400 can be inserted through the laryngeal mask component 100 and the telescopic tube component 600. At this time, the patient end of the inner tube component 400 is positioned above the patient's glottis, inside the mask body 101, and aligned with the patient end of the Laryngeal mask airway tube 102. The patient end outer connector 317 of the breathing circuit connector 310 connects to the telescopic tube machine end connector 603, and the patient end inner connector 316 of the breathing circuit connector 310 connects to the inner tube connector 403; The machine end first inner connector 318 and machine end outer connector 3110 connect to the breathing circuit, or alternatively, the machine end second inner connector 319 and machine end outer connector 3110 of the breathing circuit connector 310 connect to the breathing circuit, which connects to the anesthesia machine or ventilator, eliminating all apparatus dead space. As the telescopic tube component 600 gradually contracts, the patient end of the inner tube component 400 moves toward the patient's lungs, passing through the patient's glottis and entering the patient's trachea; When the telescopic tube component 600 contracts until it returns to its original state, the patient end of the inner tube component 400 can pass through the glottis into the trachea, thereby eliminating all apparatus dead space and part of the anatomic dead space. The third Luer taper 114 at the end of the first gas-sampling line 112 connects to the gas-sampling line of the monitor to analyze the composition and concentration of gases inhaled or exhaled by the patient. In one example, when the telescopic tube component 600 is fully extended to its completely open state, stoppers 604 are placed between the annular protrusions 605, and before the telescopic tube component 600 contracts, the stoppers 604 are removed.

When inserting a regular endotracheal tube through the laryngeal mask component 100, the connector of the endotracheal tube connects to the patient end of the connecting tube 500, the machine end of the connecting tube 500 connects to the patient end of the breathing circuit connector 310, and the second Luer taper 502 connects to the gas-sampling line of the monitor. When inserting other components of this application, such as the endotracheal tube component 200, through the laryngeal mask component 100, the inner tube component 400 is simultaneously inserted into the second ventilation cavity 206. The endotracheal tube connector 203 and the inner tube connector 403 jointly connect to the patient end of the breathing circuit connector 310. The fourth Luer taper 211 at the end of the second gas-sampling line 209 of this application connects to the gas-sampling line of the monitor.

### Embodiment 2

Referring to FIG. 1, FIG. 4-FIG. 17a, and FIG. 25, FIG. 25a, the airway management system shown in the figures comprises a laryngeal mask component 100, a breathing circuit connector component 300, an inner tube component 400, a connecting tube 500, and a telescopic tube component 600.

As shown in FIG. 17, the laryngeal mask component 100 comprises a mask body 101, a Laryngeal mask airway tube 102, a laryngeal mask airway connector 103, a laryngeal mask inflation tube 104, and a laryngeal mask inflation valve 105. One end of the Laryngeal mask airway tube 102 is connected to the mask body 101, and the other end is connected to the laryngeal mask airway connector 103. The laryngeal mask inflation tube 104 is positioned on the exterior of the Laryngeal mask airway tube 102, with one end connected to the mask body 101 for inflation, and the other end of the laryngeal mask inflation tube 104 is connected to the laryngeal mask inflation valve 105. Different from embodiment 1, the Laryngeal mask airway tube 102 of this embodiment has four internal chambers, namely the first ventilation cavity 106 which can be used for inserting the inner tube body 401, the first gas sampling cavity 108, the first throat aspiration cavity 107, and the drainage cavity 110, as shown in FIG. 17a. The first ventilation cavity 106, the first gas sampling cavity 108, the first throat aspiration cavity 107, and the drainage cavity 110 are placed inside the Laryngeal mask airway tube 102, with the patient end of the first gas sampling cavity 108 and the patient end of the first throat aspiration cavity 107 located near the patient end of the Laryngeal mask airway tube 102. The first gas sampling cavity 108 is connected to the first gas-sampling line 112, and the first throat aspiration cavity 107 is connected to the first throat aspiration tube 113. The other ends of the first gas-sampling line 112 and the first throat aspiration tube 113 are connected to a third Luer taper 114. The third Luer taper 114 connects to a monitor for analyzing the composition and concentration of gases inhaled or exhaled by the patient. The first throat aspiration tube 113 can serve as a suction channel for pharyngeal secretions from the patient, and can also function as a backup gas-sampling line.

In this embodiment, the breathing circuit connector component 300, inner tube component 400, connecting tube 500, and telescopic tube component 600 have similar structures and functions to the breathing circuit connector component 300, inner tube component 400, connecting tube 500, and telescopic tube component 600 in the aforementioned embodiment 1, which will not be repeated here.

When using the airway management system in this embodiment, it comprises the usage/working methods mentioned in the previous embodiment 1, and the identical parts will not be repeated here. The difference from the usage/working method of embodiment 1 is: during anesthesia maintenance, after the patient is inserted with the laryngeal mask component 100, the patient end of the drainage cavity 110 is connected to the patient's esophageal opening to drain the patient's gastric contents, and a gastric tube can be inserted along the drainage cavity 110.

### Embodiment 3

Please refer to FIG. 1, FIG. 4-FIG. 6 and FIG. 18-FIG. 18a, the airway management system shown in the figures comprises a laryngeal mask component 100, a breathing circuit connector component 300, an inner tube component 400, a connecting tube 500, and a telescopic tube component 600.

As shown in FIG. 18 and FIG. 18a, the laryngeal mask component 100 comprises a mask body 101, a Laryngeal mask airway tube 102, a laryngeal mask airway connector 103, a laryngeal mask inflation tube 104, and a laryngeal mask inflation valve 105. One end of the Laryngeal mask airway tube 102 is connected to the mask body 101, and the other end is connected to the laryngeal mask airway connector 103. The laryngeal mask inflation tube 104 is positioned on the exterior of the Laryngeal mask airway tube 102, with one end connected to the mask body 101 for inflation, and the other end of the laryngeal mask inflation tube 104 is connected to the laryngeal mask inflation valve 105. Different from Embodiments 1-2, the Laryngeal mask airway tube 102 of this embodiment has six internal chambers, namely a first ventilation cavity 106 for inserting the inner tube body 401, a first gas sampling cavity 108, a first throat aspiration cavity 107, a video component cavity 109, a drainage cavity 110, and a standby reserved cavity 111, as shown in FIG. 18a. The first ventilation cavity 106 is located in the central part of the Laryngeal mask airway tube 102 to facilitate the insertion of the inner tube component 400. The first gas sampling cavity 108, first throat aspiration cavity 107, video component cavity 109, drainage cavity 110, and standby reserved cavity 111 are respectively placed inside the Laryngeal mask airway tube 102, with the patient end of the first gas sampling cavity 108 and the patient end of the first throat aspiration cavity 107 positioned close to the patient end of the Laryngeal mask airway tube 102. The first gas sampling cavity 108 is connected to the first gas-sampling line 112, and the first throat aspiration cavity 107 is connected to the first throat aspiration tube 113. The other ends of the first gas-sampling line 112 and the first throat aspiration tube 113 are connected to a third Luer taper 114. The third Luer taper 114 connects to a monitor for analyzing the composition and concentration of gases inhaled or exhaled by the patient. The first throat aspiration tube 113 can serve as a suction channel for pharyngeal secretions from the patient, and can also function as a backup gas-sampling line.

The breathing circuit connector component 300, inner tube component 400, connecting tube 500, and telescopic tube component 600 in this embodiment have similar structures and functions to the breathing circuit connector component 300, inner tube component 400, connecting tube 500, and telescopic tube component 6 in the aforementioned embodiments 1-2, which will not be elaborated here.

When in use, the airway management system in this embodiment comprises the usage/working methods from the aforementioned Embodiment 2, and the identical parts will not be repeated. The difference from the usage/working method of Embodiment 2 is: during anesthesia maintenance, after the patient is inserted with the laryngeal mask component 100, a video device can be inserted through the video component cavity 109 and connected to a display, which can be used for: (1) observing the position of the mask body 101, the status of the glottis, and the condition of pharyngeal secretions, thereby making appropriate interventions. (2) When the patient end of the inner tube component 400 moves toward the patient's lungs, it can enter the trachea through the glottis under video monitoring guidance, avoiding injure to the glottis and trachea. (3) When inserting the endotracheal tube through the Laryngeal mask component 100, it can be performed under video monitoring guidance, avoiding glottis and trachea injury. The patient end of the video component cavity 109 can be cleaned through the standby reserved cavity 111, maintaining clarity and transparency at the patient end of the video component cavity 109.

### Embodiment 4

Please refer to FIG. 4-FIG. 4a, FIG. 7-FIG. 11, FIG. 19-FIG. 21, FIG. 25-FIG. 25a, which show an airway management system comprising an endotracheal tube component 200, a breathing circuit connector component 300, an inner tube component 400, and a connecting tube 500.

As shown in FIG. 19-FIG. 21, the endotracheal tube component 200 comprises a cuff 201, an endotracheal tube 222, an endotracheal tube connector 203, a cuff inflation tube 204, and a cuff inflation valve 205. The endotracheal tube 202 has four cavities, namely the second ventilation cavity 206, the second throat aspiration cavity 207, the cuff inflation cavity 212, and the second gas sampling cavity 208. The internal diameter of the second ventilation cavity 206 and of the endotracheal tube 202 are equal in each section of the lumen or are designed to have the same internal diameter; in other words, the internal diameter/size of the second ventilation cavity 206 and the endotracheal tube 202 is the same at all points. The patient end of the second gas sampling cavity 208 and the patient end of the second throat aspiration cavity 207 are close to the patient end of the endotracheal tube 202. The machine end of the cuff inflation cavity 212 is connected to the cuff inflation tube 204, and the cuff inflation tube 204 is connected to the cuff inflation valve 205. The second ventilation cavity 206 is located in the central part of the endotracheal tube 202, to facilitate the insertion of the inner tube component 400. The cuff inflation cavity 212, the second gas sampling cavity 208, and the second throat aspiration cavity 207 are placed inside the endotracheal tube 202. The second throat aspiration cavity 207 is connected to the second throat aspiration tube 210, the second gas sampling cavity 208 is connected to the second gas-sampling line 209, and the machine ends of the second throat aspiration tube 210 and the second gas-sampling line 209 are connected to the fourth Luer taper 211. The fourth Luer taper 211 connects to the monitor, used for analyzing the composition and concentration of gases inhaled or exhaled by the patient. The second throat aspiration tube 210 can serve as an aspiration channel for secretions from the throat of the patient, and can also function as a backup gas-sampling line.

The endotracheal tube 202 can have an internal diameter ranging from 2 to 20 millimeters. In some examples, the internal diameter of the endotracheal tube 202 can be any integer between 2 millimeters and 20 millimeters, and can also be: 2.5 millimeters, 3.5 millimeters, 4.5 millimeters, 5.5 millimeters, 6.5 millimeters, 7.5 millimeters, 8.5 millimeters, 9.5 millimeters, 10.5 millimeters, 11.5 millimeters, 12.5 millimeters, 13.5 millimeters, 14.5 millimeters, 15.5 millimeters, 16.5 millimeters, 17.5 millimeters, 18.5 millimeters, 19.5 millimeters, etc. The internal diameter range of the inner tube body 401 can be 2 to 12 millimeters. In some examples, the internal diameter of the inner tube body 401 can be any integer between 2 millimeters and 12 millimeters, and can also be: 2.5 millimeters, 3.5 millimeters, 4.5 millimeters, 5.5 millimeters, 6.5 millimeters, 7.5 millimeters, 8.5 millimeters, 9.5 millimeters, 10.5 millimeters, 11.5 millimeters, etc.

As shown in FIG. 7-FIG. 11, the breathing circuit connector component 300 comprises a breathing circuit connector 310, a breathing circuit connector top end cover 320, and a first Luer taper 330. The breathing circuit connector 310 comprises the patient end of the breathing circuit connector 310 and the machine end of the breathing circuit connector 310. The patient end of the breathing circuit connector 310 comprises a patient end inner connector 316 and a patient end outer connector 317. The machine end of the breathing circuit connector 310 comprises a machine end first inner connector 318, a machine end second inner connector 319, and a machine end outer connector 3110; The patient end of the breathing circuit connector 310 is equipped with a patient end inner channel 311 and a patient end outer channel 312, where the patient end inner channel 311 and patient end outer channel 312 adopt a coaxial inner and outer dual-channel structure. The machine end of the breathing circuit connector 310 contains three channels, namely the machine end first inner channel 313 located within the machine end first inner connector 318, the machine end second inner channel 314 within the machine end second inner connector 319, and the machine end outer channel 315 within the machine end outer connector 3110; The first inner channel 313, the second inner channel 314, and the inner channel 311 are connected. The outer channel 315 and the outer channel 312 are connected. An openable breathing circuit connector top end cover 320 is installed at the top of the second inner connector 319 at the machine end of the breathing circuit connector 310. The first Luer taper 330 is connected to the center of the breathing circuit connector top end cover 320. During clinical use, the patient end outer connector 317 connects to the endotracheal tube connector 203, and the patient end inner connector 316 connects to the inner tube connector 403. The machine end first inner connector 318 and the machine end outer connector 3110 connect to the breathing circuit, which connects to the anesthesia machine or ventilator. When the breathing circuit connector top end cover 320 is removed, devices such as a fiberoptic bronchoscope, closed suction tube, or bronchial occluder can be inserted through the machine end second inner channel 314; Alternatively, the machine end second inner connector 319 and the machine end outer connector 3110 can be connected to the breathing circuit. Remove the breathing circuit connector top end cover 320 and place it at the machine end first inner connector 318, with the breathing circuit connected to the anesthesia machine or ventilator.

As shown in FIG. 4-FIG. 4a and FIG. 20-FIG. 21, the inner tube component 400 comprises an inner tube body 401 and an inner tube connector 403, with a third ventilation cavity 402 set inside the inner tube body 401. According to clinical needs, the inner tube component 400, closed suction tube, or bronchial occluder, etc., can be inserted into the second ventilation cavity 206; After inserting the inner tube component 400, a closed suction tube or bronchial occluder can also be inserted into the inner tube body 401. The outer wall of the inner tube body 401 has multiple symmetrically arranged protrusions 404 along the axial direction, which are used for positioning the inner tube body 401 and the second ventilation cavity 206 inside the endotracheal tube 202.

As shown in FIG. 25-FIG. 25a, the connecting tube 500 has an unequal diameter structure, comprising a connecting tube body 501 and a second Luer taper 502. In clinical use, one end of the connecting tube body 501 connects to the patient end of the breathing circuit connector 310, and the other end connects to the anaesthetic mask. The second Luer taper 502 connects to the gas-sampling line of the monitor, which is used for anesthesia induction.

During clinical implementation of anesthesia induction, the connecting tube 5 connects the patient end of the breathing circuit connector 310 to the anaesthetic mask, and the second Luer taper 502 connects to the gas-sampling line of the monitor. The anaesthetic mask covers the patient's nose and mouth, and the breathing circuit connector 310 connects to the anesthesia machine or ventilator through the breathing circuit.

During anesthesia maintenance, after the patient is inserted with the endotracheal tube component 200, the endotracheal tube connector 203 connects to the patient end of the breathing circuit connector 310, and the machine end of the breathing circuit connector 310 connects to the anesthesia machine or ventilator through the breathing circuit. The fourth Luer taper 211 of the second gas-sampling line 209 connects to the monitor gas-sampling line, analyzing the composition and concentration of gases inhaled or exhaled by the patient. The machine end first inner connector 318 and machine end outer connector 3110 connect to the breathing circuit, or alternatively, the machine end second inner connector 319 and machine end outer connector 3110 connect to the breathing circuit, which connects to the anesthesia machine or ventilator. The machine end first inner connector 318 and machine end outer connector 3110 of the breathing circuit connector 310 connect to the breathing circuit, when the breathing circuit connects to the anesthesia machine or ventilator, a fiberoptic bronchoscope, closed suction tube, bronchial occluder, etc. can be inserted through the machine end second inner channel 314.

During anesthesia maintenance, after the patient is inserted with the endotracheal tube component 200, an inner tube component 400 can also be inserted through the endotracheal tube 202 and endotracheal tube connector 203. The patient end of the inner tube component 400 aligns with the patient end of the endotracheal tube 202, while the endotracheal tube connector 203 and the inner tube connector 403 jointly connect to the patient end of the breathing circuit connector 310. The patient end outer connector 317 connects to the endotracheal tube connector 203, and the patient end inner connector 316 connects to the inner tube connector 403. The machine end first inner connector 318 and machine end outer connector 3110 connect to the breathing circuit, or alternatively, the machine end second inner connector 319 and machine end outer connector 3110 connect to the breathing circuit. The breathing circuit connects to the anesthesia machine or ventilator, which can eliminate all apparatus dead space. The fourth Luer taper 211 at the end of the second gas-sampling line 209 connects to the monitor gas-sampling line, analyzing the composition and concentration of gases inhaled or exhaled by the patient.

During anesthesia maintenance, after the patient is inserted with the endotracheal tube component 200, the endotracheal tube connector 203 connects to the patient end of the breathing circuit connector component 300, then remove the breathing circuit connector top end cover 320 and place it on the machine end first inner connector 318. The inner tube body 401 is inserted through the machine end second inner connector 319, the inner tube connector 403 and the machine end outer connector 3110 are connected to the patient end of the breathing circuit respectively, while the machine end of the breathing circuit connects to the anesthesia machine or ventilator. As the inner tube body 401 is gradually inserted, the apparatus dead space within the airway management system gradually decreases, and when the patient end of the inner tube component 400 aligns with the patient end of the endotracheal tube 202, all apparatus dead space is eliminated. The patient end of the inner tube component 400 can continue to be inserted into the trachea, thereby eliminating all apparatus dead space plus part of the anatomic dead space.

### Embodiment 5

Please refer to FIG. 4-FIG. 4a, FIG. 7-FIG. 11 and FIG. 22-FIG. 25a, which show an airway management system, comprising an endotracheal tube component 200, a breathing circuit connector component 300, an inner tube component 400, and a connecting tube 500.

As shown in FIG. 22-FIG. 24, the endotracheal tube component 200 comprises a cuff 201, an endotracheal tube 202, an endotracheal tube connector 203, a cuff inflation tube 204, and a cuff inflation valve 205. The endotracheal tube component 200 has four cavities inside the endotracheal tube 202, namely the second ventilation cavity 206, the second throat aspiration cavity 207, the cuff inflation cavity 212, and the second gas sampling cavity 208. Different from Embodiment 4, in this embodiment, the second ventilation cavity 206 and the endotracheal tube 202 are designed with a structure where the internal diameter of the section below the patient's glottis is smaller than the internal diameter of the section above the patient's glottis. In other words, the internal diameters/dimensions of the second ventilation cavity 206 and the endotracheal tube 202 vary at different locations, with the internal diameter/dimension of the section positioned below the patient's glottis during use being smaller than the internal diameter/dimension of the section positioned above the patient's glottis during use. The patient end of the second gas sampling cavity 208 and the patient end of the second throat aspiration cavity 207 are close to the patient end of the endotracheal tube 202. The machine end of the cuff inflation cavity 212 is connected to the cuff inflation tube 204, and the cuff inflation tube 204 is connected to the cuff inflation valve 205. The second ventilation cavity 206 is located in the central part of the endotracheal tube 202, to facilitate the insertion of the inner tube component 400. The cuff inflation cavity 212, the second gas sampling cavity 208, and the second throat aspiration cavity 207 are placed inside the endotracheal tube 202. The second throat aspiration cavity 207 is connected to the second throat aspiration tube 210, the second gas sampling cavity 208 is connected to the second gas-sampling line 209, and the machine ends of the second throat aspiration tube 210 and the second gas-sampling line 209 are connected to the fourth Luer taper 211. The fourth Luer taper 211 connects to the monitor, used for analyzing the composition and concentration of gases inhaled and exhaled by the patient. The second throat aspiration tube 210 can serve as an aspiration channel for secretions from the throat of the patient, and can also function as a backup gas-sampling line.

The breathing circuit connector component 300, inner tube component 400, and connecting tube 500 in this embodiment have similar structures and functions to the breathing circuit connector component 300, inner tube component 400, and connecting tube 500 in the aforementioned Embodiment 4, which will not be elaborated here.

During anesthesia maintenance, after the patient is inserted with the endotracheal tube component 200, the endotracheal tube connector 203 is connected to the patient end of the breathing circuit connector 310, and the machine end of the breathing circuit connector 310 is connected to the anesthesia machine or ventilator via breathing circuit. The fourth Luer taper 211 of the second gas-sampling line 209 connects to the monitor gas-sampling line, analyzing the composition and concentration of gases inhaled or exhaled by the patient. The machine end first inner connector 318 and machine end outer connector 3110 connect to the breathing circuit, or alternatively, the machine end second inner connector 319 and machine end outer connector 3110 connect to the breathing circuit, which connects to the anesthesia machine or ventilator. The machine end first inner connector 318 and machine end outer connector 3110 connect to the breathing circuit, when the breathing circuit connects to the anesthesia machine or ventilator, a fiberoptic bronchoscope, closed suction tube, bronchial occluder, etc. can be inserted through the machine end second inner channel 314.

During anesthesia maintenance, after the patient is inserted with the endotracheal tube component 200, an inner tube component 400 can also be inserted through the endotracheal tube 202 and endotracheal tube connector 203. The patient end of the inner tube component 4 is positioned at the patient end of the larger internal diameter portion of the endotracheal tube 202. The endotracheal tube connector 203 and the inner tube connector 403 jointly connect to the patient end of the breathing circuit connector 310. The patient end outer connector 317 connects to the endotracheal tube connector 203, and the patient end inner connector 316 connects to the inner tube connector 403; The machine end first inner connector 318 and machine end outer connector 3110 connect to the breathing circuit, or alternatively, the machine end second inner connector 319 and machine end outer connector 3110 connect to the breathing circuit. The breathing circuit connects to the anesthesia machine or ventilator, which can eliminate part of the apparatus dead space. The fourth Luer taper 211 at the end of the second gas-sampling line 209 connects to the monitor gas-sampling line, analyzing the composition and concentration of gases inhaled or exhaled by the patient.

During anesthesia maintenance, after the patient is inserted with the endotracheal tube component 200, the endotracheal tube connector 203 connects to the patient end of the breathing circuit connector component 300, then remove the breathing circuit connector top end cover 320 and place it on the machine end first inner connector 318. The inner tube body 401 is inserted through the machine end second inner connector 319, the inner tube connector 403 and the machine end outer connector 3110 are connected to the patient end of the breathing circuit respectively. As the inner tube body 401 is gradually inserted, the apparatus dead space within the airway management system gradually decreases. When the patient end of the inner tube component 400 is positioned at the patient end of the larger diameter portion of the endotracheal tube 202, part of the apparatus dead space is eliminated.

The endotracheal tube 202 and endotracheal tube connector 203 or Laryngeal mask airway tube 102 and laryngeal mask airway connector 103 of this application have larger internal diameters than existing products, resulting in lower artificial airway resistance compared to existing products when patients have large spontaneous breathing tidal volumes. This application allows for the insertion of an inner tube component 400 through the endotracheal tube 202 and endotracheal tube connector 203 or through the Laryngeal mask airway tube 102 and laryngeal mask airway connector 103, which can reduce or even eliminate apparatus dead space. After inserting the inner tube component 400, the third ventilation cavity 402 and the second ventilation cavity 206, or the third ventilation cavity 402 and the first ventilation cavity 106, respectively form inhalation and exhalation cavities that can be interchanged. Which cavity serves as the inhalation cavity and which serves as the exhalation cavity can be determined according to clinical needs. The patient end of the gas sampling cavity is closer to the alveoli and is not affected by dead space gas, making sampling analysis more sensitive and accurate. The laryngeal mask airway connector 103 can connect to the patient end of the telescopic tube component 600. When the telescopic tube component 600 is fully extended to its completely open state, a stopper 604 is placed between the annular protrusions 605 of the telescopic tube component 600. The inner tube component 400 can be inserted through the laryngeal mask component 100 and the telescopic tube component 600, which can eliminate the apparatus dead space. After removing the stopper 604, as the telescopic tube component 600 gradually contracts, the patient end of the inner tube component 400 moves toward the patient's lungs, passing through the patient's glottis and entering the patient's trachea; When the telescopic tube component 600 is contracted to its original state, the patient end of the inner tube component 400 reaches the patient's trachea, eliminating all apparatus dead space and part of the anatomic dead space.

The embodiments described in the drawings of this application are exemplary, only used to explain this application, and should not be understood as limitations on this application.

What is claimed is:

1. An airway management system, comprising a laryngeal mask component and an inner tube component, and a third ventilation cavity is arranged inside the inner tube body; the laryngeal mask component comprises a mask body, a laryngeal mask airway tube connected to one end of the mask body, a first ventilation cavity is arranged inside the laryngeal mask airway tube, and the first ventilation cavity is configured for inserting the inner tube component;
the patient end of the inner tube component is configured to be positioned within the mask body, and aligned with the patient end of the laryngeal mask airway tube; The patient end of the inner tube component can also be configured to enter the trachea of the patient through the glottis of the patient; after inserting the inner tube component, between the first ventilation cavity and the third ventilation cavity, one forms an inhalation cavity and another forms an exhalation cavity.

2. The airway management system according to claim 1, wherein between the ventilation cavity formed after the first ventilation cavity inserted into the inner tube component and the third ventilation cavity, one forms an inhalation cavity and another forms an exhalation cavity; or
between the part of the first ventilation cavity inserted into the inner tube component and the third ventilation cavity, one forms an inhalation cavity and another forms an exhalation cavity.

3. The airway management system according to claim 1, wherein the laryngeal mask component further comprises a laryngeal mask connector, where the other end of the laryngeal mask airway tube is connected to the laryngeal mask connector; the inner tube component also comprises an inner tube connector connected to the inner tube body; the airway management system also comprises a breathing circuit connector component; the breathing circuit connector component comprises a breathing circuit connector; one end of the breathing circuit connector is connected to the laryngeal mask connector, or connected to the laryngeal mask connector and the inner tube connector, and the other end of the breathing circuit connector is configured to connect to an anesthesia machine or a ventilator through a breathing circuit;
the breathing circuit connector comprises a patient end of the breathing circuit connector, the patient end of the breathing circuit connector comprises a patient end inner connector and a patient end outer connector, wherein the patient end outer connector is configured to connect to the laryngeal mask connector, the patient end inner connector is configured to connect to the inner tube connector.

4. The airway management system according to claim 3, wherein the airway management system further comprises a telescopic tube component, the telescopic tube component comprises a telescopic tube patient end connector, a telescopic tube machine end connector, and a telescopic tube body, one end of the telescopic tube body is connected to the telescopic tube patient end connector, the other end of the telescopic tube body is connected to the telescopic tube machine end connector; the telescopic tube patient end connector is configured to connect the laryngeal mask connector; the inner tube component is configured to be inserted via the laryngeal mask airway tube, the laryngeal mask connector and the telescopic tube component; the telescopic tube machine end connector and the inner tube connector are configured to connect to the patient end of the breathing circuit connector;
when the telescopic tube component is configured to fully extend to its completely open stage, the patient end of the inner tube component reaches the trachea of the patient; when the telescopic tube component gradually contracts, the patient end of the inner tube component moves towards the lungs of the patient, passes through the glottis of the patient, and enters the trachea of the patient;
wherein the telescopic tube component further comprises: annular protrusions and a stopper; the annular protrusions are provided on outer side walls of the telescopic tube patient end connector and the telescopic tube machine end connector, respectively; the stopper is configured to limit the telescopic tube component; and when the telescopic tube component is fully extended to its completely open stage, the stopper is located between the annular protrusions.

5. The airway management system according to claim 1, wherein a first gas sampling cavity, and a first throat aspiration cavity are further provided in the laryngeal mask airway tube, wherein a patient end of a first gas sampling cavity and a patient end of a first throat aspiration cavity are close to the patient end of the laryngeal mask airway tube, the first gas sampling cavity inside the laryngeal mask airway tube and the first throat aspiration cavity inside the laryngeal mask airway tube are fixedly mated.

6. The airway management system according to claim 3, wherein the machine end of the breathing circuit connector comprises a machine end first inner connector, a machine end second inner connector, and a machine end outer connector; the machine end of the breathing circuit connector is provided with three channels, the three channels being a machine end first inner channel, a machine end second inner channel, and a machine end outer channel, wherein the patient end of the breathing circuit connector comprises a patient end inner channel and a patient end outer channel, the machine end first inner channel and the machine end second inner channel is connected to the patient end inner channel, the machine end outer channel is connected to the patient end outer channel, wherein the patient end inner channel and the patient end outer channel are of a coaxial inner and outer dual-channel structure, the machine end first inner connector and the machine end outer connector or the machine end second inner connector and the machine end outer connector are connected to an anesthesia machine or a ventilator through the breathing circuit, respectively;
wherein the breathing circuit connector component further comprises a breathing circuit connector top end cover, the openable breathing circuit connector top end cover is provided at the top end of the machine end second inner connector, and the breathing circuit connector top end cover is configured to be connected to the machine end first inner connector;
wherein the breathing circuit connector component further comprises a first Luer taper; and the middle of the breathing circuit connector top end cover is connected to the first Luer taper.

7. The airway management system according to claim 3, wherein the airway management system further comprises a connecting tube, and the connecting tube is of an unequal diameter structure, wherein the connecting tube comprises a connecting tube body; when clinical anesthesia induction is performed, one end of the connecting tube body is configured to be connected to the patient end of the breathing circuit connector, and the other end of the connecting tube body is configured to be connected to an anesthetic mask.

8. The airway management system according to claim 5, wherein a drainage cavity is further provided in the laryngeal mask airway tube;
wherein a video component cavity is further provided in the laryngeal mask airway tube;
wherein a standby reserved cavity is further provided in the laryngeal mask airway tube.

9. The airway management system according to claim 1, wherein an outer wall of the inner tube body is provided with a plurality of protrusions; the protrusion is configured for positioning between the inner tube body and the first ventilation cavity or the second ventilation cavity.

10. The airway management system according to claim 6, wherein the connecting tube further comprises a second Luer taper, the second Luer taper is configured to be connected to a gas sampling line of a monitor.

11. An airway management system, comprising an endotracheal tube component and an inner tube component, wherein the inner tube component comprises an inner tube body, a third ventilation cavity is arranged inside the inner tube body, the endotracheal tube component comprises an endotracheal tube, a second ventilation cavity is arranged in the endotracheal tube, the second ventilation cavity is configured for inserting the inner tube component; after inserting the inner tube component, between the second ventilation cavity and the third ventilation cavity, one forms an inhalation cavity and another forms an exhalation cavity;
wherein the endotracheal tube has the same internal diameter; the patient end of the inner tube component is configured to be align with the patient end of the endotracheal tube; the patient end of the inner tube component is also configured to enter the trachea of the patient through the patient end of the endotracheal tube; or
the internal diameter of the endotracheal tube is configured such that the internal diameter of the segment located below the glottis of a patient when in use is smaller than that of the segment located above the glottis of the patient when in use; the patient end of the inner tube component is configured to be located at the patient end of the large diameter portion of the endotracheal tube of the unequal diameter structure.

12. The airway management system according to claim 11, wherein the ventilation cavity formed after the second ventilation cavity is inserted into the inner tube component and the third ventilation cavity, one forms an inhalation cavity and another forms an exhalation cavity; or
the portion of the second ventilation cavity inserted into the inner tube component and the third ventilation cavity, one forms an inhalation cavity, and another forms an exhalation cavity.

13. The airway management system according to claim 11, wherein the endotracheal tube component further comprises an endotracheal tube connector, the inner tube component comprises an inner tube connector connected to the inner tube body, the airway management system further comprises a breathing circuit connector component, the breathing circuit connector component comprises a breathing circuit connector, one end of the breathing circuit connector is connected to the endotracheal tube connector or to the endotracheal tube connector and the inner tube connector, the other end of the breathing circuit connector is configured to be connected to an anesthesia machine or ventilator through breathing circuit;
the breathing circuit connector comprises a patient end of the breathing circuit connector, the patient end of the breathing circuit connector comprises a patient end inner connector and a patient end outer connector, the patient end outer connector is configured to be connected to the endotracheal tube connector, the patient end inner connector is configured to be connected to the inner tube connector.

14. The airway management system according to claim 13, wherein the machine end of the breathing circuit connector comprises a machine end first inner connector, a machine end second inner connector, and a machine end outer connector; the machine end of the breathing circuit connector is provided with three channels, the three channels being a machine end first inner channel, a machine end second inner channel, and a machine end outer channel; the patient end of the breathing circuit connector comprises a patient end inner channel, and a patient end outer channel, the machine end first inner channel and the machine end second inner channel is connected to the patient end inner channel, the machine end outer channel is connected to the patient end outer channel, the patient end inner channel and the patient end outer channel are of a coaxial inner and outer dual-channel structure, the machine end first inner connector and the machine end outer connector or the machine end second inner connector and the machine end outer connector are connected to an anesthesia machine or a ventilator through the breathing circuit, respectively.

15. The airway management system according to claim 14, wherein the breathing circuit connector component further comprises a breathing circuit connector top end cover, the openable breathing circuit connector top end cover is provided at the top end of the machine end second inner connector, and the breathing circuit connector top end cover is configured to be connected to the machine end first inner connector;
wherein the breathing circuit connector component further comprises a first Luer taper; and the middle of the breathing circuit connector top end cover is connected to the first Luer taper.

16. The airway management system according to claim 13, wherein the airway management system further comprises a connecting tube, and the connecting tube is of an unequal diameter structure, wherein the connecting tube comprises a connecting tube body; when clinical anesthesia induction is performed, one end of the connecting tube body is configured to be connected to the patient end of the breathing circuit connector, and the other end of the connecting tube body is configured to be connected to an anesthetic mask.

17. The airway management system according to claim 16, wherein the connecting tube further comprises a second Luer taper, the second Luer taper is configured to be connected to a gas sampling line of a monitor.

18. The airway management system according to claim 11, wherein an outer wall of the inner tube body is provided with a plurality of protrusions; the protrusion is configured for positioning between the inner tube body and the first ventilation cavity or the second ventilation cavity.

19. The airway management system according to claim 11, wherein a second throat aspiration cavity, a second gas sampling cavity, and a cuff inflation cavity are provided in the endotracheal tube.

20. The airway management system according to claim 19, wherein the patient end of the second gas sampling cavity and the patient end of the second throat aspiration cavity are close to the patient end of the endotracheal tube. The second gas sampling cavity and the second throat aspiration cavity are fixedly mated.
